(19) United States
(12) Patent Application Publication
(10) Pub. No.: US 2014/0056951 A1
(43) Pub. Date: Feb. 27, 2014

(54) METHODS AND COMPOSITIONS FOR TREATING BIOFILMS

(71) Applicant: PRESIDENT AND FELLOWS OF HARVARD COLLEGE, Cambridge, MA (US)

(72) Inventors: Richard LOSICK, Lexington, MA (US); Illana KOLODKIN-GAL, Cambridge, MA (US); Jon CLARDY, Jamaica Plain, MA (US); Shugeng CAO, Waltham, MA (US); Matt CABEEN, Dorchester, MA (US); Roberto KOLTER, Cambridge, MA (US); Liraz CHAI, Giv`atayim (IL); Thomas BÖTTCHER, Maisach (DE)

(73) Assignee: PRESIDENT AND FELLOWS OF HARVARD COLLEGE, Cambridge, MA (US)

(21) Appl. No.: 14/070,785

(22) Filed: Nov. 4, 2013

Related U.S. Application Data
(63) Continuation of application No. PCT/US2012/036662, filed on May 4, 2012.
(60) Provisional application No. 61/482,523, filed on May 4, 2011.
(60) Provisional application No. 61/482,522, filed on May 4, 2011.
(60) Provisional application No. 61/591,601, filed on Jan. 27, 2012.

(51) Int. Cl. A61K 31/4025; C07C 211/09; C07C 211/11; C07C 217/42; C07D 259/00; C07D 207/34; C07D 207/337; C07C 237/06; A61K 31/198; A61K 31/132; A61K 31/137; A61K 31/40; A61K 31/164; A61K 45/06; C07C 211/13

(52) U.S. Cl. CPC A61K 31/4025; C07C 211/13; C07C 211/09; C07C 211/11; C07C 217/42; C07D 259/00; C07D 207/34; C07D 207/337; C07C 237/06; A61K 31/198; A61K 31/132; A61K 31/137; A61K 31/40; A61K 31/164; A61K 45/06
USPC 424/400; 514/674; 514/673; 514/670; 514/183; 514/423; 514/427; 514/614; 514/567; 424/54

(57) ABSTRACT

Methods of treating or reducing biofilms, treating a biofilm-related disorder, and preventing biofilm formation using polyamines is described.

### CROSS REFERENCE TO RELATED APPLICATIONS

This application is a continuation of International Application No. PCT/US12/36662, filed May 4, 2012, which claims the benefit of U.S. Provisional Application No. 61/591,601, filed Jan. 27, 2012, U.S. Provisional Application No. 61/482,523, filed May 4, 2011, and U.S. Provisional Application No. 61/482,522, filed May 4, 2011, the disclosures of all of which are hereby incorporated by reference herein.

### STATEMENT OF GOVERNMENT RIGHTS

This invention was made with Government support under National Institutes of Health awards GM18568, CA24487, GM86258, GM058213, GM082137, and AI057159. The Government has certain rights in the invention.

### BACKGROUND

Biofilms are communities of cells that settle and proliferate on surfaces and are covered by an exopolymer matrix. They are slow-growing and many are in the stationary phase of growth. A hallmark of biofilms is an extracellular matrix typically consisting of protein, exopolysaccharide and sometimes DNA, that holds the cells together in the community. They can be formed by most, if not all, pathogens. According to the CDC, 65% of all infections in the United States are caused by biofilms that can be formed by common pathogens. Biofilms are also found in industrial settings, such as in drinking water distribution systems.

### SUMMARY

It has been discovered that certain polyamines inhibit biofilm formation and trigger biofilm disassembly. Aspects of this disclosure feature methods of treating, reducing, inhibiting biofilm formation by bacteria, and triggering biofilm disassembly. In some embodiments, the method comprises contacting a surface with a composition comprising an effective amount of a polyamine, thereby treating, reducing, inhibiting formation of the biofilm, or triggering disassembly of the biofilm.

It has been discovered that the structure of the polyamine contributes to its ability to inhibit biofilm formation and trigger disassembly of a biofilm. In certain embodiments, the polyamine has at least three amino groups separated by three atoms either in a straight chain or cyclic molecule. In certain embodiments, the amino groups are ionizable. In certain embodiments, the amino groups are positively charged. In some embodiments, the polyamines are branched. In some embodiments, the polyamines are linear. In certain embodiments, the polyamine has Formula (I),

HR₇N-(M-N(R₇)—)ₓ-M-N(R₇)—Y—N(R₇)-(M-N(R₇)—)ₓ-M-NR₇H (I)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

In some embodiments, the polyamines can have Formula (Ia), where some R₇ groups in Formula (I) is replaced with R₇ₐ, which is defined as H, C₁₋₈ ω-amino alkyl, C₂₋₈ ω-amino alkenyl, C₂₋₈ ω-amino alkynyl, amino alkaryl, amino aryl, N-heteroaryl. In such cases, the polyamines have Formula (Ia),

HR₇N-(M-N(R₇ₐ)—)ₓ-M-N(R₇)—Y—N(R₇ₐ)-(M-N(R₇ₐ)—)ₓ-M-NR₇H (Ia)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

each R₇ₐ is H, C₁₋₈ ω-amino alkyl, C₂₋₈ ω-amino alkenyl, C₂₋₈ ω-amino alkynyl, amino alkaryl, amino aryl, N-heteroaryl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

Compounds of Formulae (I) may be acyclic or cyclic. If a compound of Formulae (I) is cyclic, the terminal NHR₁ groups of Formula (I) are NR₁ and form a ring where each NR₁ covalently bonds to one M group, thereby forming a ring.

In certain embodiments, the polymer or oligomer blocks of Y can comprise one or more of carbonyl, epoxy, ester, carboxyl, amine, amide, imine, imide, or glycol.

In some embodiments, the polyamine has Formula (II),

Hₐ-[R₇N-(L-N(R₈)—)ₓ-L-NH—Pd]_{c} (II),

wherein

each P is R₇ or Q or L;

each L is M or —C(R₁R₂)—X—C(R₅R₆)— or

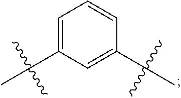 [SMILES: *c1cccc(*)c1]

each M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;

each R₈ is H, C₁₋₈ co-amino alkyl, C₂₋₈ ω-amino alkenyl, C₂₋₈ ω-amino alkynyl, amino alkaryl, amino aryl, N-heteroaryl;

each X is —NH—, —O—, —N(R₈)—, or S;

each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;

a is 0 or 1, wherein when a is 0 the polyamine is cyclic and when a is 1 the polyamine is linear or branched;

each b is 0 or 1;

c is greater than or equal to 1; and

each x is greater than or equal to 1.

In some embodiments of Formula (II), a is 1 and the polyamine is linear or branched. In certain embodiments of Formula (II), a is 1, b is 1, c is 1, L is M, P is R₇, R₇ and R₈ are hydrogen, and the polyamine has Formula (IIa),

H₂N-(M-NH)ₓ-M-NH₂ (IIa).

In certain embodiments a is 0 and compounds of Formula (II) may be cyclic. In some embodiments, a is 0, b is 1, P is Q, and the ring is formed by Q bonding to the terminal NR₇ group. In some embodiments, a is 0, b is 1, P is L, L is M, R₇ and R₈ are hydrogen, the terminal NR₇ group (where R₇ is hydrogen) bonds to the terminal L group (which is M), and the cyclic polyamine has Formula (IIb),

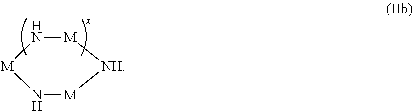

Compounds of Formulae (I), (Ia), (II), and (IIa) may be linear or branched.

In some embodiments of compounds of Formulae (I), (Ia), (II), (IIa), and (IIb), x may be 1. In some embodiments of compounds of Formulae (I), (Ia), (II), (IIa), and (IIb), x may be 2. In other embodiments of compounds of the above Formulae (I), (Ia), (II), (IIa), and (IIb), x may be 3. In still other embodiments of compounds of the above Formulae (I), (Ia), (II), (IIa), and (IIb), x may be 4. In still other embodiments of compounds of the above Formulae (I), (Ia), (II), (IIa), and (IIb), x may be 5. In further embodiments of compounds of the above Formulae (I), (Ia), (II), (IIa), and (IIb), x may be greater than 5.

In some embodiments, M is —CH₂CH₂CH₂—. In some embodiments, R₇ is —(CH₂)₁₁CH₃. In other embodiments, R₇ is hydrogen. In some embodiments, R₈ is hydrogen. In other embodiments, R₈ is —(CH₂)₃NH₂.

In some embodiments, at least one of R₁, R₂, R₃, R₄, R₅, or R₆ is C₁alkenyl and the other R group attached to the same carbon atom does not exist. For example, if R₁ is C₁-alkenyl, then R₂ does not exist.

In some embodiments, L is

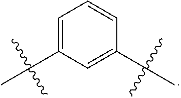 [SMILES: *c1cccc(*)c1]

Compound i in Table 1 exemplifies such an embodiment.

In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane. In certain embodiments, the polyamine has Formulae (I), (Ia), (II), (IIa), or (IIb). In other embodiments, the polyamine is a compound in Table 1.

In some embodiments, the bacteria are Gram-negative or Gram-positive bacteria. In particular embodiments, the bacteria are Bacillus, Staphylococcus, E. coli, or Pseudomonas bacteria. In some embodiments, the bacteria are mycobacteria.

In certain embodiments, the method comprises contacting a surface with a composition comprising an effective amount of a polyamine combined with a D-amino acid, thereby treating, reducing, inhibiting formation of the biofilm, or triggering disassembly of the biofilm.

In other aspects, this disclosure features compositions, such as industrial, therapeutic or pharmaceutical compositions, comprising one or more polyamines.

In other aspects, the invention features compositions, such as therapeutic or pharmaceutical compositions, comprising one or more polyamines. In some embodiments, the one or more polyamines are combined with one or more D-amino acids. In some embodiments, the composition comprises at least one polyamine of Formulae (I), (Ia), (II), (IIa), or (IIb). In some embodiments, the polyamine has Formulae (I), (Ia), (II), (IIa), or (IIb) and the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the D-amino acid is D-tyrosine or the combination of D-amino acids comprises D-tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

Another aspect of this disclosure is directed to methods of treating a biofilm-related disorder in a subject in need thereof, the method comprising administering to the subject a composition comprising an effective amount of a polyamine. In some embodiments, the polyamine is norspermidine, norspermine, 1,5,9-triazacyclododecane, or a combination thereof. In certain embodiments, the polyamine has Formulae (I), (Ia), (II), (IIa), or (IIb). In other embodiments, the polyamine is a compound in Table 1.

In some embodiments, the method comprises administering to the subject a composition comprising a combination of at least one polyamine and at least one D-amino acid, thereby treating the biofilm-related disorder. In some embodiments, the polyamine has Formulae (I), (Ia), (II), (IIa), or (IIb) and the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the D-amino acid is D-tyrosine or the combination of D-amino acids comprises D-tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the composition is administered to a surface of the subject selected from the group of dermal and mucosal surfaces and combinations thereof. In other embodiments, the surface is an oral surface, a skin surface, a urinary tract surface, a vaginal tract surface, or a lung surface.

In some embodiments, the composition is administered to the subject via subcutaneous, intra-muscular, intra-peritoneal, intravenous, oral, nasal, or topical administration, and a combination thereof.

In some embodiments, the subject is a human.

In some embodiments, the formation of a biofilm is inhibited. In other embodiments, a previously formed biofilm is disrupted.

In some embodiments, the polyamine is administered at a concentration of about 0.1 nM to about 100 μM, for example, at a concentration of 0.1 nM to 100 μM. In certain embodiments, the D-amino acid combined with the polyamine is administered at a concentration of about 0.1 nM to about 100 μM, for example, at a concentration of 0.1 nM to 100 μM.

In further embodiments, the biofilm-related disorder is selected from the group consisting of pneumonia, cystic fibrosis, otitis media, chronic obstructive pulmonary disease, and a urinary tract infection and combinations thereof. In other embodiments, the biofilm-related disorder is a medical device-related infection. In further embodiments, the biofilm-related disorder is a periodontal disease, such as gingivitis, periodontitis or breath malodor. In still further embodiments, the biofilm-related disorder is caused by bacteria. In some embodiments, the bacteria are Gram-negative or Gram-positive bacteria. In still other embodiments, the bacteria are of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas.

Another aspect of this disclosure is directed to methods of treating, reducing, inhibiting biofilm formation by biofilm forming bacteria, or triggering biofilm disassembly on a biologically-related surface, the method comprising contacting a biological surface with a composition comprising an effective amount of a polyamine. In certain embodiments, the polyamine has Formulae (I), (Ia), (II), (IIa), or (IIb). In some embodiments, the polyamine is norspermidine, norspermine, 1,5,9-triazacyclododecane, or a combination thereof. In other embodiments, the polyamine is a compound in Table 1.

In certain embodiments, the composition and related methods include a combination of at least one polyamine and at least one D-amino acid, thereby treating, reducing or inhibiting formation of the biofilm. In some embodiments, the polyamine has Formulae (I), (Ia), (II), (IIa), or (IIb) and the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments of the foregoing methods, the composition comprises a polyamine and D-tyrosine. In addition to D-tyrosine, in some embodiments, the composition further comprises one or more of D-proline and D-phenylalanine. In still other embodiments, in addition to D-tyrosine, the composition further comprises one or more of D-leucine, D-tryptophan, and D-methionine. In still further embodiments, in addition to D-tyrosine, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, and D-tryptophan.

In some embodiments, the surface comprises a medical device, a wound dressing, a contact lens, or an oral device. In other embodiments, the medical device is selected from the group consisting of a clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator, and an endoscope and combinations thereof.

In some embodiments of any of the foregoing methods, the method further comprises administering a biocide. In some embodiments, the biocide is an antibiotic.

Yet another aspect of the invention is directed to compositions comprising at least one polyamine, or at least one polyamine combined with at least one D-amino acid or a mixture of D-amino acids, in an amount effective to treat, reduce, inhibit biofilm formation, or trigger biofilm disassembly. In certain embodiments, the polyamine has Formulae (I), (Ia), (II), (IIa), or (IIb) or is selected from the group of compounds in Table 1, or is a combination one or more compounds in Table 1, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof or the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the composition comprises a polyamine and D-tyrosine. In certain embodiments, the polyamine has Formulae (I), (Ia), (II), (IIa), or (IIb) or is selected from the group of compounds in Table 1, or is a combination one or more compounds in Table 1. In other embodiments the composition further comprises one or more of D-proline and D-phenylalanine. In still other embodiments, the composition further comprises one or more of D-leucine, D-tryptophan, and D-methionine. In further embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, any of the foregoing compositions can also comprise polyhexamethylene biguanide, chlorhexidine, xylitol, triclosan, or chlorine dioxide. In other embodiments, any of the foregoing compositions can also comprise a pharmaceutically acceptable carrier. In still other embodiments of any the foregoing compositions, the effective amount is an amount effective to treat or prevent a biofilm-related disorder. In some embodiments, an effective amount comprises and amount effective to treat or prevent a biofilm on a surface.

In yet other embodiments of any the foregoing compositions, the biofilm-related disorder is pneumonia, cystic fibrosis, otitis media, chronic obstructive pulmonary disease, or a urinary tract infection. In some embodiments, the biofilm-related disorder is a medical device-related infection.

In some embodiments of any of the foregoing compositions, the composition further comprises an agent suitable for application to the surface. In other embodiments of any of the foregoing compositions, the composition is formulated as a wash solution, a dressing, a wound gel, or a synthetic tissue. In further embodiments, the composition is formulated as tablets, pills, troches, capsules, aerosol spray, solutions, suspensions, gels, pastes, creams, or foams. In some embodiments, the composition is formulated for parenteral, e.g., intravenous, intradermal, subcutaneous, oral (e.g., inhalation), transdermal (topical), transmucosal, vaginal and rectal administration.

Another aspect of this disclosure is directed to biofilm resistant medical devices, comprising a surface likely to contact a biological fluid and a polyamine. In certain embodiments, the polyamine has Formulae (I), (Ia), (II), (IIa), or (IIb) or is selected from the group of compounds in Table 1. In some embodiments, the medical device further comprises a D-amino acid, or a combination of D-amino acids, combined with the polyamine and coated on or impregnated into said surface. In some embodiments, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the combination of D-amino acids is in an amount effective to treat, reduce, or inhibit biofilm formation, the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the D-amino acid is D-tyrosine or the combination of D-amino acids comprises D-tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the polyamine is formulated as a slow-release formulation. In certain embodiments, the polyamine combined with the D-amino acid is formulated as a slow-release formulation. In some embodiments, the surface is essentially free of L-amino acids. In further embodiments, the surface is essentially free of detergent.

In some embodiments, the device is selected from one or more of clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator and endoscope.

A further aspect of the instant disclosure is directed to potable liquids comprising a polyamine. In some embodiments, the polyamine has Formulae (I), (Ia), (II), (IIa), or (IIb). In some embodiments, the polyamine is selected from the group of compounds in Table 1. In other embodiments at least one polyamine is combined with at least one D-amino acid or a combination of D-amino acids, at a concentration in the range of 0.000001% to 0.1%. In some embodiments, wherein the polyamine has Formulae (I), (Ia), (II), (IIa), or (Ith) or is selected from the group of compounds in Table 1, or is a combination one or more compounds in Table 1, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the D-amino acid is D-tyrosine or the combination of D-amino acids comprises D-tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

Another aspect of this disclosure is directed to compositions resistant to biofilm formation, comprising a pharmaceutically or cosmetically suitable base, and an effective amount of a polyamine. In some embodiments, the polyamine has Formulae (I), (Ia), (II), (IIa), or (IIb). In some embodiments, the polyamine is selected from the group of compounds in Table 1. In certain embodiments, the polyamine is combined with a D-amino acid or a combination of D-amino acids, distributed in the base, thereby treating, reducing or inhibiting formation of the biofilm. In some embodiments, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments of the methods and compositions of this disclosure, the base is essentially free of the corresponding L-amino acid or L-amino acids relative to the D-amino acids or combination of D-amino acids.

In some embodiments, the base is selected from a liquid, gel, paste, or powder. In further embodiments, the composition is selected from the group consisting of shampoos, bath additives, hair care preparations, soaps, lotions, creams, deodorants, skin-care preparations, cosmetic personal care preparations, intimate hygiene preparations, foot care preparations, light protective preparations, skin tanning preparations, insect repellants, antiperspirants, shaving preparations, hair removal preparations, fragrance preparations, dental care, denture care and mouth care preparations and combinations thereof.

### BRIEF DESCRIPTION OF THE FIGURES

The following figures are presented for the purpose of illustration only, and are not intended to be limiting.

FIGS. 1A-E show the identification of norspermidine in conditioned medium from B. subtilis and the effect of norspermidine on pellicle formation.

FIG. 1A shows the result of growing cells in fresh medium to which had been added 20 μA of the 25%, 35% or 40% methanol eluates.

FIG. 1B shows the results of cells of NCBI3610 that were grown in fresh medium containing PBS buffer (control), norspermidine (100 μM), morpholine (100 μM) HPLC-purified fatty acid (˜100 μM), or spermidine (100 μM).

FIG. 1C shows the detection of norspermidine in pellicles.

FIGS. 1D & 1E shows the quantification of the biofilm-inhibiting activity of norspermidine and spermidine.

FIGS. 2A and 2B1-2B4 show the results of the testing of various concentrations of norspermidine on biofilms and the detection of norspermidine.

FIG. 2A shows the minimal biofilm inhibiting concentration of norspermidine. Pellicle formation of strain NCBI 3610 was tested in the presence of various concentrations of norspermidine as indicated.

FIG. 2B1-2B4 show the detection of norspermidine. 2B1: Norspermidine purchased from Sigma Aldrich was used a standard for the detection of norspermidine in the biofilm. Norspermidine was derivatized with Fmoc-Cl and the resulting Fmoc-norspermidine (RT: 10.1 min) was quantified using an Agilent LC/MS system. 2B2: The UV spectrum of the reaction product of Fmoc-Cl and norspermidine at 10 min. 2B3: Positive MS (798 Da) of the reaction product of Fmoc-Cl and norspermidine at 10.1 min. 2B4: Derivatization reaction of norspermidine with Fmoc-Cl.

FIG. 3A shows 7 day-old cultures of the wild type (WT), a mutant (ΔgbaT) blocked in norspermidine production (IKG623), a double mutant (ΔylmE AracX) blocked in D-amino acid production (IKG55) and a triple mutant (ΔgbaT ΔylmE ΔracX) blocked in the production of both (IKG625).

FIG. 3B show the effects of combinations of amino acids and norspermidine at indicated concentrations on biofilm formation.

FIG. 3C shows the results of quantifying the pellicle breakdown to see whether a combination of D-amino acids and norspermidine was more effective than either D-amino acids or norspermidine alone.

FIG. 4A shows that cells for a mutant for a homolog of the norspermidine decarboxylase gene yaaO are delayed in pellicle disassembly. NCBI 3610 (WT), a mutant for yaaO (IKG624), a mutant doubly deleted for ylmE and racX (IKG55) or a triple mutant for yaaO, ylmE and racX (IKG626) for were grown in 12-well plates and incubated for 7 days.

FIG. 4B shows 3-day-old cultures of the wild type (NCBI 3610), an exopolysaccharide mutant (ΔepsH; DS76), a TasA mutant (ΔtasA; FC55), and, as indicated, wild type and mutant strains grown in the presence of 25 μM norspermidine.

FIG. 5 shows phase contrast and fluorescence images of cells of the wild-type (WT; NCBI 3610) harvested from pellicles grown in the presence or absence (untreated) of norspermidine (25 μM) or a high concentration of spermidine (1 mM). The cells were washed in PBS and stained for exopolysaccharide with a conjugate of concanavalin A with Texas-Red.

FIG. 6A shows that concanavalin A-Texas Red stain is largely specific to exopolysaccharide. Fluorescence microscopy was carried out with 3-day-old standing cultures. Cells of wild type strain (NCBI 3610) and an eps mutant (DS76) were collected and stained for one hour as in Experimental Procedures. Cells were imaged using the indicated exposure times. Little or no staining was observed for the mutant except when image brightness was enhanced as shown in the enlargement or when the cells were stained for 150 minutes (data not shown). Images were collected using the automated software program SimplePC1.

FIG. 6B shows that TasA-mCherry is not released from norspermidine-treated cells. NCBI 3610 containing the tasA-mCherry fusion (DR30) was grown without shaking in a biofilm medium (upper row) or in the same medium applied with norspermidine (50 μM, lower row). Cells were washed in PBS, and visualized by fluorescence microscopy.

FIG. 7A shows the average hydrodynamic radii of the exopolysaccharide as measured by dynamic light scattering. Shown are the results obtained in the absence of polyamine (black), in the presence of norspermidine (white), and in the presence of spermidine (grey) with exopolysaccharide at the indicated concentrations and pH. Error bars represent the standard deviation of polymer radii among the polymers in a single sample.

FIG. 7B shows three different magnifications of representative fields showing exopolysaccharide alone (EPS) and exopolysaccharide that had been mixed with norspermidine (EPS+norspermidine) or with spermidine (EPS+spermidine).

FIG. 8A shows that norspermidine does not inhibit growth of B. subtilis at concentrations that block biofilm formation.

FIG. 8B shows that norspermidine does not inhibit expression of P_{epsA}-lacZ at concentrations that block biofilm formation. Strain FC5 (carrying P_{epsA}-lacZ) was grown in MSgg medium containing norspermidine (100 μM) with shaking or in untreated medium as control (NT).

FIG. 9A shows the compounds that were tested for biofilm-inhibiting activity.

FIG. 9B shows the effect of the numbered compounds on pellicle formation by NCBI3610. The compounds were tested at 200 μM.

FIGS. 9C & 9D show computer modeling of the interaction of norspermidine and spermidine with an acidic exopolysaccharide.

FIG. 10 shows the results of the examination of relationship of the structure and activity of the polyamines in B. subtilis.

FIG. 11 shows the computer modeling of norspermidine binding to a neutral exopolysaccharide. Two PGA stands can be aligned with norspermidine by alternating hydrogen bonds to the acetyl groups of PGA.

FIG. 12A shows the effect of the numbered compounds displayed in FIG. 9A on the formation of submerged biofilms by S. aureus strain SCO1. The compounds were tested at 500 μM. Biofilm formation was visualized by crystal violet staining of submerged biofilms.

FIG. 12B shows quantification of the effects of norspermidine, norspermine, spermine and spermidine as measured by crystal violet staining (see Experimental procedures).

FIG. 13A shows the effect of the numbered compounds shown in FIG. 9A on submerged biofilm formation by E. coli strain MC4100. The compounds were tested at 500 μM. Biofilm formation was visualized by crystal violet staining of submerged biofilms.

FIG. 13B shows quantification of the effects of norspermidine, norspermine, spermine and spermidine as measured by crystal violet staining (see Experimental procedures).

FIG. 14A shows the results of the examination of the relationship of the structure and activity of the polyamines in S. aureus. The effect of the numbered compounds on the formation of submerged biofilms by S. aureus strain SCO1. Biofilm formation was visualized by crystal violet staining of submerged biofilms.

FIG. 14B shows the results of the examination of the relationship of the structure and activity of the polyamines in E. coli. The effect of the numbered compounds on the formation of submerged biofilms by E. coli strain MC4100. Biofilm formation was visualized by crystal violet staining of submerged biofilms.

FIG. 15 shows the inhibition of biofilm formation Bacillus subtilis in cells treated with (B) norspermidine and (E) norspermine as compared to the formation of biofilms in (A) cells not treated (“NT”) or treated with (C) spermidine or (D) speramide.

FIG. 16 is photographs of shows that cells treated with norspermidine (B) and norspermine (C) inhibit pellicle formation by B. subtilis as compared with untreated cells (A) and those treated with spermidine (D) or spermine (E).

FIGS. 17A and 17B shows that polyamines mediate pellicle disassembly by B. subtilis by comparison of an untreated cell culture (A) having an intact pellicle and a culture treated with norspermine (B) showing a disrupted pellicle.

FIGS. 18A, 18B, 18C, and 18D show that 1,5,9-triazacyclododecane inhibits biofilm formation by B. subtilis. FIGS. 18A and 18C are not treated with the polyamine. FIGS. 18B and 18D are treated with 1,5,9-triazacyclododecane.

FIG. 19AH shows that polyamines inhibit biofilm formation by Staphylococcus aureus.

FIGS. 20A-D shows that polyamines act synergistically with D-amino acids in inhibiting biofilm formation by Staphylococcus.

FIGS. 21A-H shows that polyamines inhibit biofilm formation by Pseudomonas aeruginosa.

FIG. 22 shows that polyamines inhibit biofilm formation by Proteus mirabilis. Biofilm formation was visualized by crystal violet staining of submerged biofilms.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims. As will be apparent to one of skill in the art, specific features and embodiments described herein can be combined with any other feature or embodiment.

### Definitions

The term “alkyl” refers to a hydrocarbon chain that may be a straight chain or branched chain. The chain may contain an indicated number of carbon atoms. For example, C₁-C₁₂ indicates that the group may have from 1 to 12 (inclusive) carbon atoms in it. An alkyl group can be substituted or unsubstituted. When substituted, one or more carbon atoms may be replaced with a heteroatom such as N, O, or S.

The term alkoxy refers to a straight or branched chain saturated or unsaturated hydrocarbon containing at least one oxygen atom. The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy include, but are not limited to, methoxy, ethoxy, isopropoxy, butoxy, n-pentoxy, isopentoxy, neopentoxy, and hexoxy. An alkoxy group can be substituted or unsubstituted. When substituted, one or more carbon atoms may be replaced with a heteroatom such as N, O, or S.

The term alkenyl refers to a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. Exemplary such groups include, but are not limited to, ethenyl (also called “vinyl”), allyl, propenyl, crotyl, 2-isopentenyl, allenyl, butenyl, butadienyl, pentenyl, pentadienyl, 3(1,4-pentadienyl), hexenyl and hexadienyl. When the indicated number of carbon atoms is 1, then the C₁ alkenyl is double bonded to a carbon. An alkenyl group can be substituted or unsubstituted. When substituted, one or more carbon atoms may be replaced with a heteroatom such as N, O, or S.

The term alkynyl refers to a straight or branched chain hydrocarbon radical containing at least one carbon-carbon triple bond. The chain may contain an indicated number of carbon atoms. For example, “C₂-C₁₂ alkynyl” indicates that the group may have from 2 to 12 (inclusive) carbon atoms and at least one carbon-carbon triple bond. Exemplary such groups include, but are not limited to, ethynyl, propynyl and butynyl. An alkynyl group can be substituted or unsubstituted. When substituted, one or more carbon atoms may be replaced with a heteroatom such as N, O, or S.

The term “aryl” refers to cyclic aromatic carbon ring systems containing from 6 to 18 carbons. Examples of an aryl group include, but are not limited to, phenyl, naphthyl, anthracenyl, tetracenyl, and phenanthrenyl. An aryl group can be unsubstituted or substituted. When substituted, one or more carbon atoms may be replaced with a heteroatom such as N, O, or S.

The term “aralkyl” refers to an alkyl group where an H has been replaced with an aryl group. An aralkyl group may be unsubstituted or it may be substituted on the hydrocarbon chain or the aryl ring. When substituted, one or more carbon atoms may be replaced with an N, O, or S.

The term “heteroaryl” refers to mono and bicyclic aromatic groups of 4 to 10 atoms containing at least one heteroatom. Heteroatom as used in the term heteroaryl refers to oxygen, sulfur and nitrogen. A heteroaryl group can be unsubstituted or substituted.

The terms “disorder”, “disease”, and “condition” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. A disorder or disease can refer to a biofilm-related disorder that is characterized by a disease-related growth of bacteria in that a biofilm is established.

The terms “prevent,” “preventing,” and “prevention” refer herein to the inhibition of the development or onset of a biofilm or of a biofilm-related disorder or the prevention of the recurrence, onset, or development of one or more indications or symptoms of a biofilm or of a biofilm-related disorder on a surface or in a subject resulting from the administration of a composition described herein (e.g., a prophylactic or therapeutic composition), or the administration of a combination of therapies (e.g., a combination of prophylactic or therapeutic compositions).

As used herein, “treat”, “treating” or “treatment” refers to administering a composition described herein in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a biofilm or with a biofilm-related disorder or an indication or symptom thereof, e.g., to a statistically significant degree or to a degree detectable to one skilled in the art. An effective amount, manner, or mode can vary depending on the surface, application, and/or subject and may be tailored to the surface, application, and/or subject. By preventing or slowing progression of a biofilm or of a biofilm-related disorder or an indication or symptom thereof, a treatment can prevent or slow deterioration resulting from a biofilm or from a biofilm-related disorder or an indication or symptom thereof on an effected surface or in an affected or diagnosed subject.

### Polyamines

Polyamines are small organic compounds found in most cells. Polyamines putrescine (1,4-diaminobutane), spermidine (1,8-diamino-4-azaoctane) and spermine (1,12-diamino-4,9-diazaoctane) are required in micromolar to millimolar concentrations to support a wide variety of cellular functions. Depletion of polyamines can result in disruption of cellular functions and can cause cytotoxicity. For example, spermidine and spermine promote biofilm formation in some bacteria. It has therefore been surprisingly discovered that certain polyamines inhibit biofilm formation and/or disassemble existing biofilms.

This disclosure is based, at least in part, on the discovery that polyamines present in conditioned medium from mature biofilms inhibit biofilm formation and trigger the disassembly of existing biofilms. As shown in the Examples, it was discovered that the biofilm-inhibiting effect of norspermidine was specific in that a closely related polyamine, spermidine (differing only by an extra methylene group), exhibited little activity. Similarly, another polyamine, norspermine, was also active in biofilm inhibition whereas its close relative spermine (once again, having an extra methylene) was inactive. These discoveries, coupled with the results shown in the Examples, led to the development of the polyamines described herein that can inhibit biofilm formation and trigger biofilm disassembly. Polyamines discovered to be particularly suitable for use as biofilm inhibitors include polyamines comprising propylamino units and whose amino units are ionizable.

Without being bound by theory, it is believed that norspermidine acts to disrupt or inhibit biofilm by targeting the exopolysaccharide. There are several pieces of evidence, which are shown in the Examples, that support this. First, norspermidine and D-amino acids acted cooperatively in inhibiting biofilm formation, suggesting that they function by different mechanisms. Second, pellicles formed in the presence of norspermidine resembled the wispy, fragmented material produced by an exopolysaccharide mutant but not the thin, flat, featureless pellicle of a mutant blocked in amyloid-fiber production. Third, fluorescence microscopy showed that norspermidine (but not spermidine) disrupted the normal uniform pattern of staining of exopolysaccharide but had little effect on the staining pattern of the protein component of the matrix. Finally, light scattering and electron microscopy experiments revealed that norspermidine, but not spermidine, interacted with purified exopolysaccharide.

Remarkably, the biofilm-inhibiting effect of norspermidine and norspermine was not limited to B. subtilis. Both molecules inhibited the formation of submerged biofilms by S. aureus and E. coli. Indeed, the same pattern of molecules that were active or inactive in inhibiting biofilm formation by B. subtilis was observed for S. aureus and E. coli. Therefore, the polyamines described herein use a common mechanism of targeting the exopolysaccharide. Indeed, this was supported by fluorescence microscopy with S. aureus and E. coli and light scattering experiments with purified exopolysaccharide from E. coli.

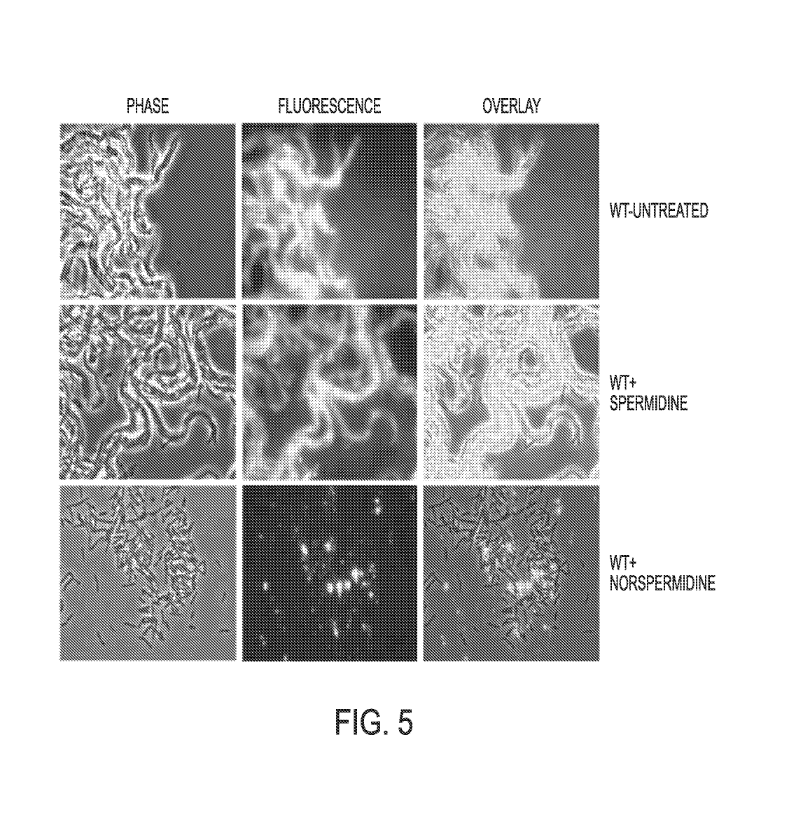

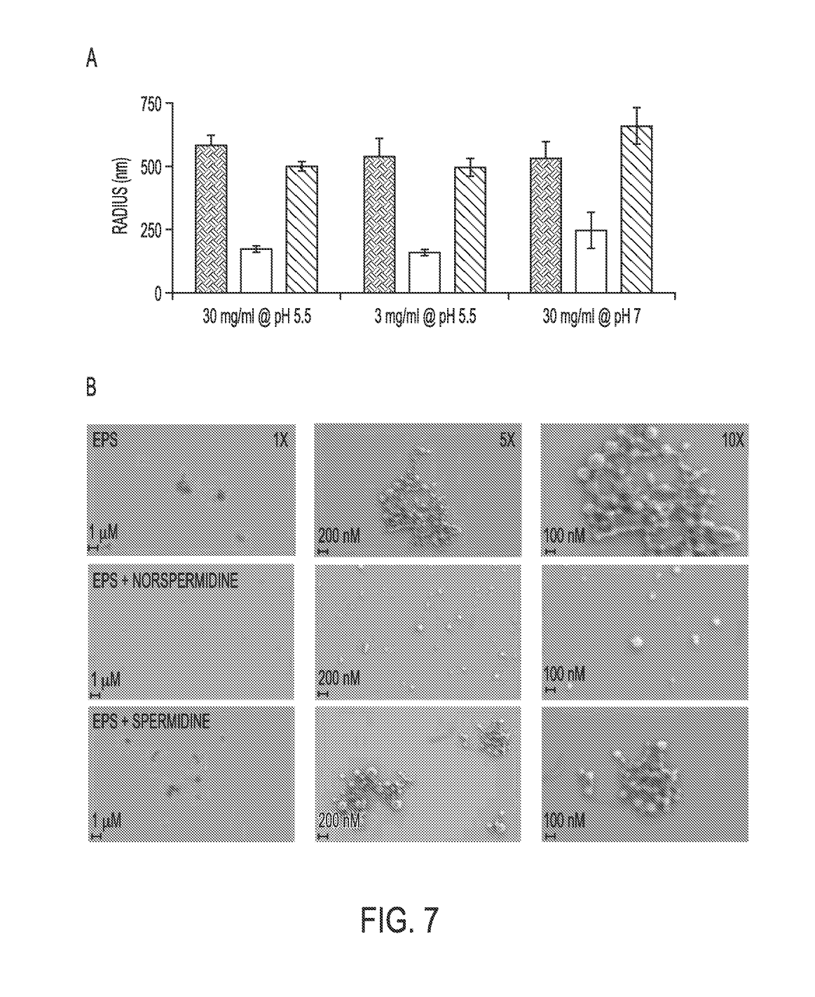

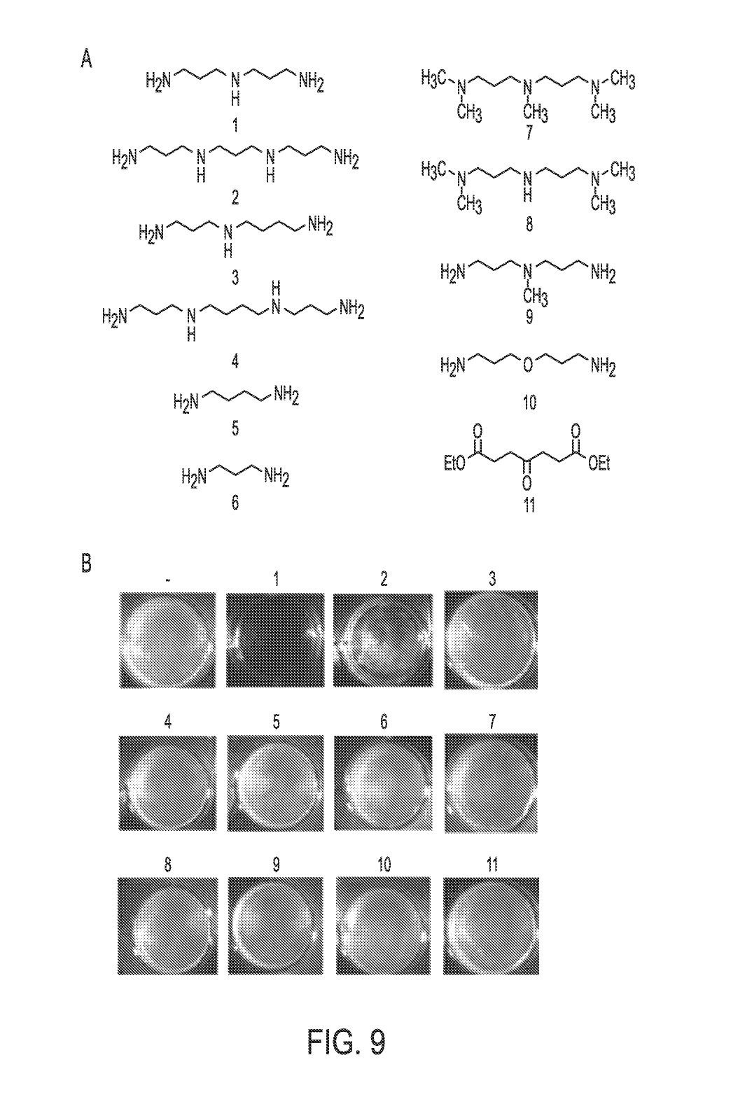

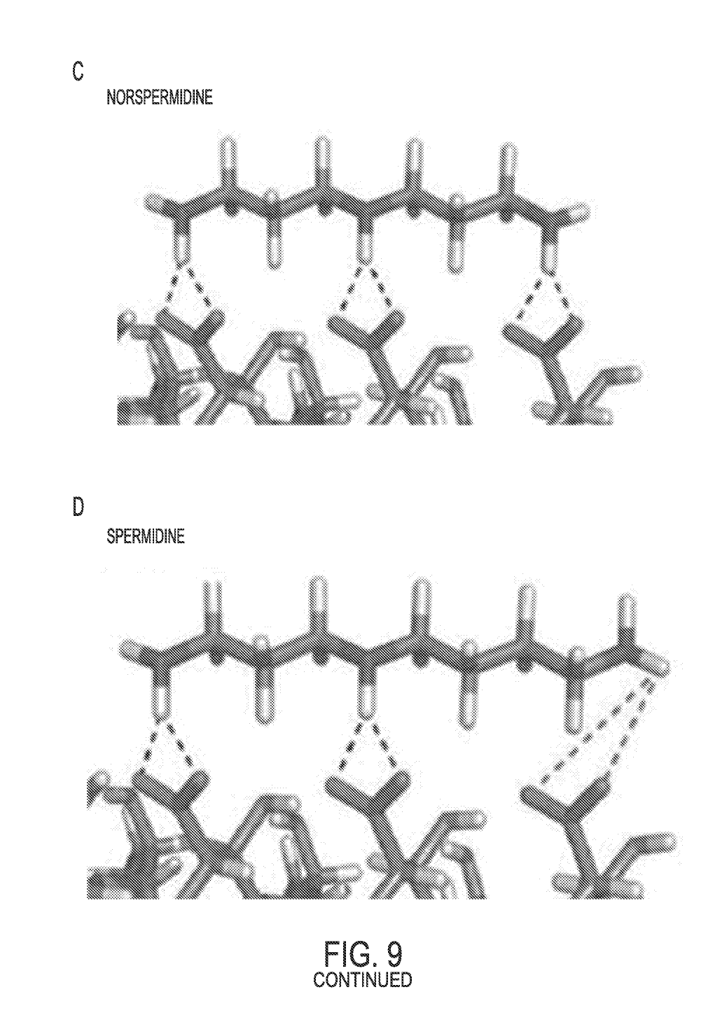

Exopolysaccharides often contain negatively charged residues (e.g. uronic acid) or neutral sugars with polar groups (e.g. poly-N-acetylglucosamine). Molecular modeling suggests that the amines in norspermidine, but not those in spermidine, are capable of interacting with such charged (FIGS. 9C and 9D) or polar groups (FIG. 11) in secondary structure of the exopolysaccharide. This interaction enhances the ability of the exopolysaccharide polymers to interact with each other or with other parts of the polymer chain. Indeed, the results of fluorescence microscopy (FIG. 5), dynamic light scattering (FIG. 7A), and scanning electron microscopy (FIG. 7B) indicate that the exopolysaccharide network collapses upon addition of norspermidine. Without being bound by theory, it is possible that exopolysaccharide polymers form an interwoven meshwork in the matrix that helps hold cells together and that condensation of the polymers in response to norspermidine weakens the meshwork and causes release of polymers.

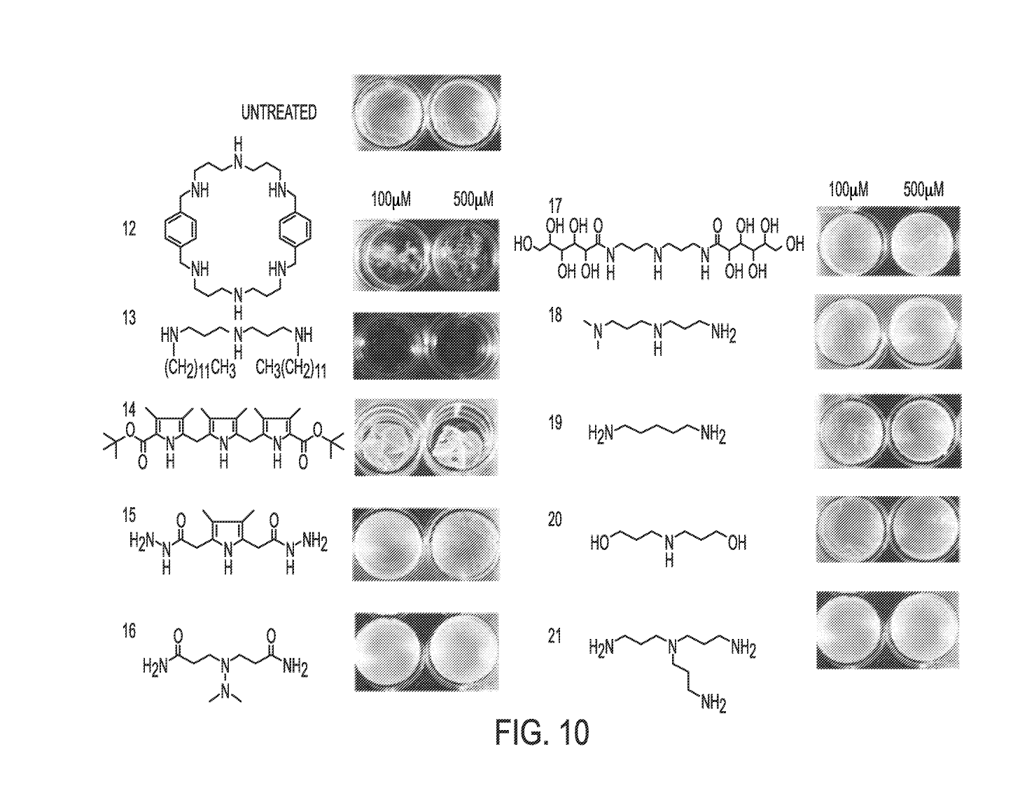

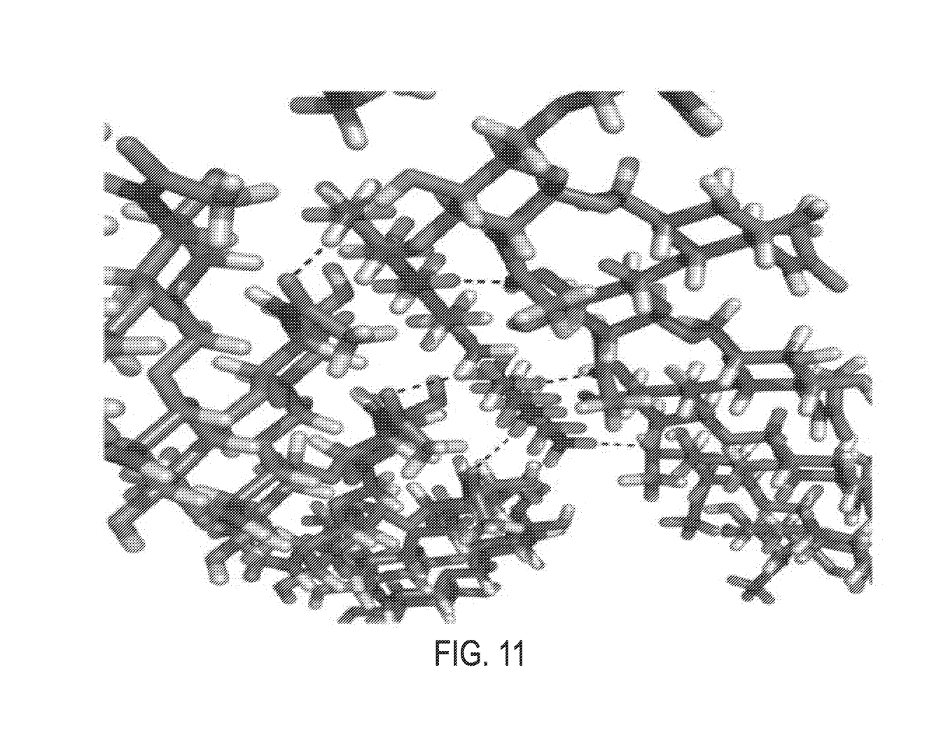

Furthermore, as seen in the examples, it was also discovered that the charge of each amine (at the neutral pH of the medium) was also important for biofilm inhibiting activity. (See Example 6). Molecules that had neutral amide bonds instead of amines separated by three methylenes (15-17) were only weakly active or inactive (FIG. 10 and Table 2). The more effective polyamines have amino groups that are ionizable. This ionizable feature of the polyamines further supports the theory that the polyamines target the exopolysaccharide.

Given the apparent versatility of norspermidine and norspermine in inhibiting biofilm formation by a variety of bacteria, the polyamines described in this application were developed to interact with the exopolysaccharides of biofilms and prevent biofilm formation by medically and industrially important microorganisms.

In some aspects, this disclosure features compositions, such as therapeutic or pharmaceutical compositions, comprising one or more polyamines. In certain embodiments, the polyamine has at least three amino groups separated by three atoms either in a straight chain or cyclic molecule. In certain embodiments, the polyamine has Formula (I),

HR₇N-(M-N(R₇)—)ₓ-M-N(R₇)—Y—N(R₇)-(M-N(R₇)—)ₓ-M-NR₇H (I)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

In some embodiments, the polyamines can have Formula (Ia), where some R₇ groups in Formula (I) is replaced with R₇ₐ, which is defined as H, C₁₋₈ ω-amino alkyl, C₂₋₈ ω-amino alkenyl, C₂₋₈ ω-amino alkynyl, amino alkaryl, amino aryl, N-heteroaryl. In such cases, the polyamines have Formula (Ia),

HR₇N-(M-N(R₇ₐ)—)ₓ-M-N(R₇)—Y—N(R₇ₐ)-(M-N(R₇ₐ)—)ₓ-M-NR₇H (Ia)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

each R₇ₐ is H, C₁₋₈ ω-amino alkyl, C₂₋₈ ω-amino alkenyl, C₂₋₈ ω-amino alkynyl, amino alkaryl, amino aryl, N-heteroaryl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

Compounds of Formulae (I) may be acyclic or cyclic. If a compound of Formulae (I) is cyclic, the terminal NHR₁ groups of Formula (I) are NR₁ and form a ring where each NR₁ covalently bonds to one M group, thereby forming a ring.

In certain embodiments, the polymer or oligomer blocks of Y can comprise one or more of carbonyl, epoxy, ester, carboxyl, amine, amide, imine, imide, or glycol.

In some embodiments, the polyamine has Formula (II),

Hₐ—[R₇N-(L-N(R₈)—)ₓ-L-NH—Pd]_{c} (II),

wherein

each P is R₇ or Q or L;

each L is M or —C(R₁R₂)—X—C(R₅R₆)— or

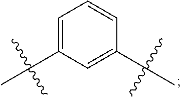 [SMILES: *c1cccc(*)c1]

each M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;

each R₈ is H, C₁₋₈ ω-amino alkyl, C₂₋₈ ω-amino alkenyl, C₂₋₈ ω-amino alkynyl, amino alkaryl, amino aryl, N-heteroaryl;

each X is —NH—, —O—, —N(R₈)—, or S;

each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;

a is 0 or 1, wherein when a is 0 the polyamine is cyclic and when a is 1 the polyamine is linear or branched;

each b is 0 or 1;

c is greater than or equal to 1; and

each x is greater than or equal to 1.

In some embodiments of Formula (II), a is 1 and the polyamine is linear or branched. In certain embodiments of Formula (II), a is 1, b is 1, c is 1, L is M, P is R₇, R₇ and R₈ are hydrogen, and the polyamine has Formula (IIa),

H₂N-(M-NH)ₓ-M-NH₂ (IIa).

In certain embodiments a is 0 and compounds of Formula (II) may be cyclic. In some embodiments, a is 0, b is 1, P is Q, and the ring is formed by Q bonding to the terminal NR₇ group. In some embodiments, a is 0, b is 1, P is L, L is M, R₇ and R₈ are hydrogen, the terminal NR₇ group (where R₇ is hydrogen) bonds to the terminal L group (which is M), and the cyclic polyamine has Formula (IIb),

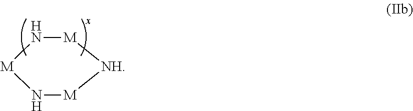

Compounds of Formulae (I), (Ia), (II), and (IIa) may be linear or branched.

In some embodiments of compounds of Formulae (I), (Ia), (II), (IIa), and (IIb), x may be 1. In some embodiments of compounds of Formulae (I), (Ia), (II), (IIa), and (IIb), x may be 2. In other embodiments of compounds of the above Formulae (I), (Ia), (II), (IIa), and (IIb), x may be 3. In still other embodiments of compounds of the above Formulae (I), (Ia), (II), (IIa), and (IIb), x may be 4. In still other embodiments of compounds of the above Formulae (I), (Ia), (II), (IIa), and (IIb), x may be 5. In further embodiments of compounds of the above Formulae (I), (Ia), (II), (IIa), and (IIb), x may be greater than 5.

In some embodiments, M is —CH₂CH₂CH₂—. In some embodiments, R₇ is —(CH₂)₁₁CH₃. In other embodiments, R₇ is hydrogen. In some embodiments, R₈ is hydrogen. In other embodiments, R₈ is —(CH₂)₃NH₂.

In some embodiments, at least one of R₁, R₂, R₃, R₄, R₅, or R₆ is C₁alkenyl and the other R group attached to the same carbon atom does not exist. For example, if R₁ is C₁-alkenyl, then R₂ does not exist.

In some embodiments, L is

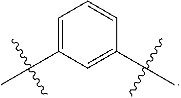 [SMILES: *c1cccc(*)c1]

Compound i in Table 1 exemplifies such an embodiment.

In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane. In certain embodiments, the polyamine has Formulae (I), (Ia), (II), (IIa), or (IIb). In other embodiments, the polyamine is a compound in Table 1.

TABLE 1

| Compound | Formula |
|---|---|
| a | 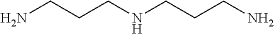 Norspermidine (also known as N-(3-aminopropyl)propane- 1,3-diamine) |
| b | 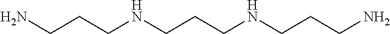 [SMILES: NCCCNCCCNCCCN] Norspermine |
| c | 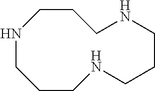 [SMILES: C1CNCCCNCCCNC1] 1,5,9-triazacyclododecane |
| d | 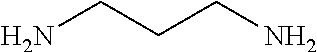 1,3-diaminopropane |
| e | 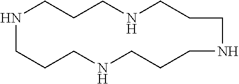 [SMILES: C1CNCCCNCCCNCCCNC1] 1,5,9,13-tetraazacyclohexadecane |
| f | 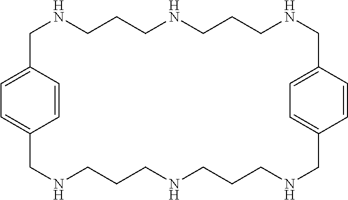 [SMILES: c1cc2ccc1CNCCCNCCCNCc1ccc(cc1)CNCCCNCCCNC2] 3,7,11,18,22,26-Hexaazatricyclo[26.2.2.213,16] tetratriaconta-13,15,28,30,31,33-hexaene |
| g | 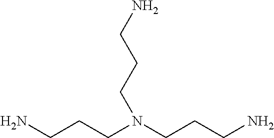 [SMILES: NCCCN(CCCN)CCCN] N¹,N¹-bis(3-aminopropyl)propane-1,3-diamine |
| h | 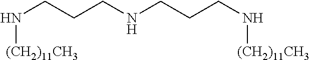 N1-dodecyl-N3-(3-(dodecylamino)propyl)propane- 1,3-diamine |
| i | 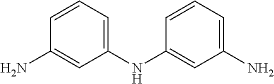 [SMILES: Nc1cccc(Nc2cccc(N)c2)c1] N,N-Di(3-aminophenyl)amine |

In some embodiments, compositions of the present disclosure include a compound from Table 1, or a combination of one or more compounds from Table 1. In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane.

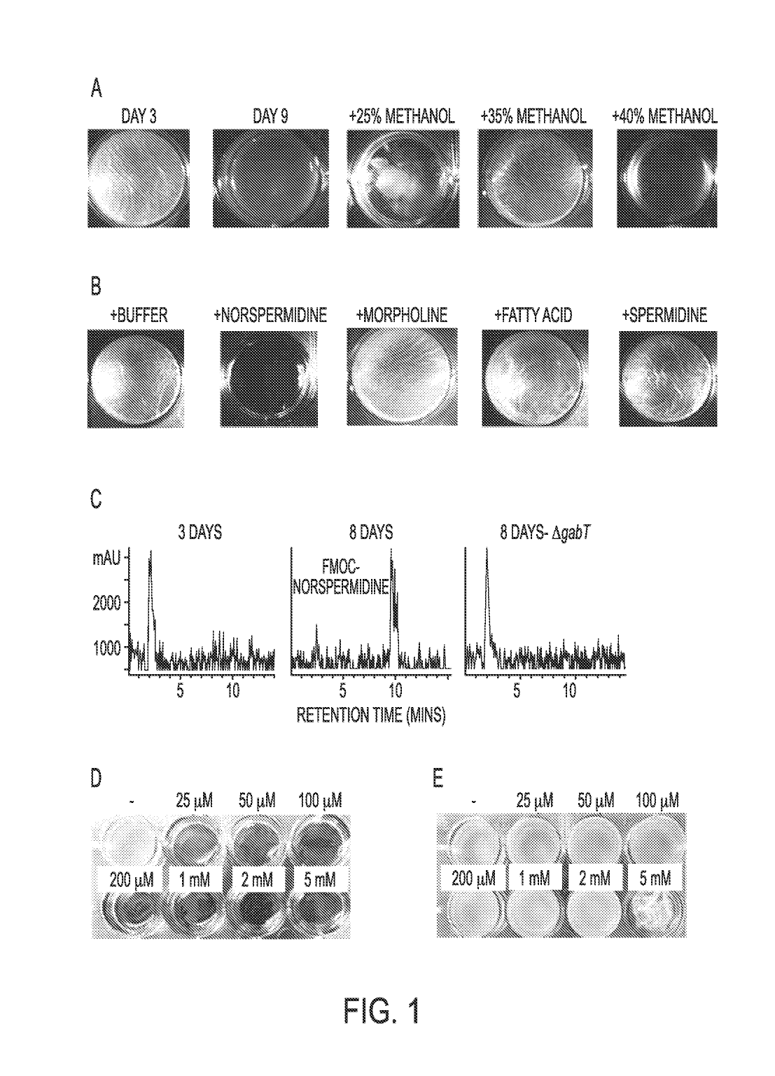

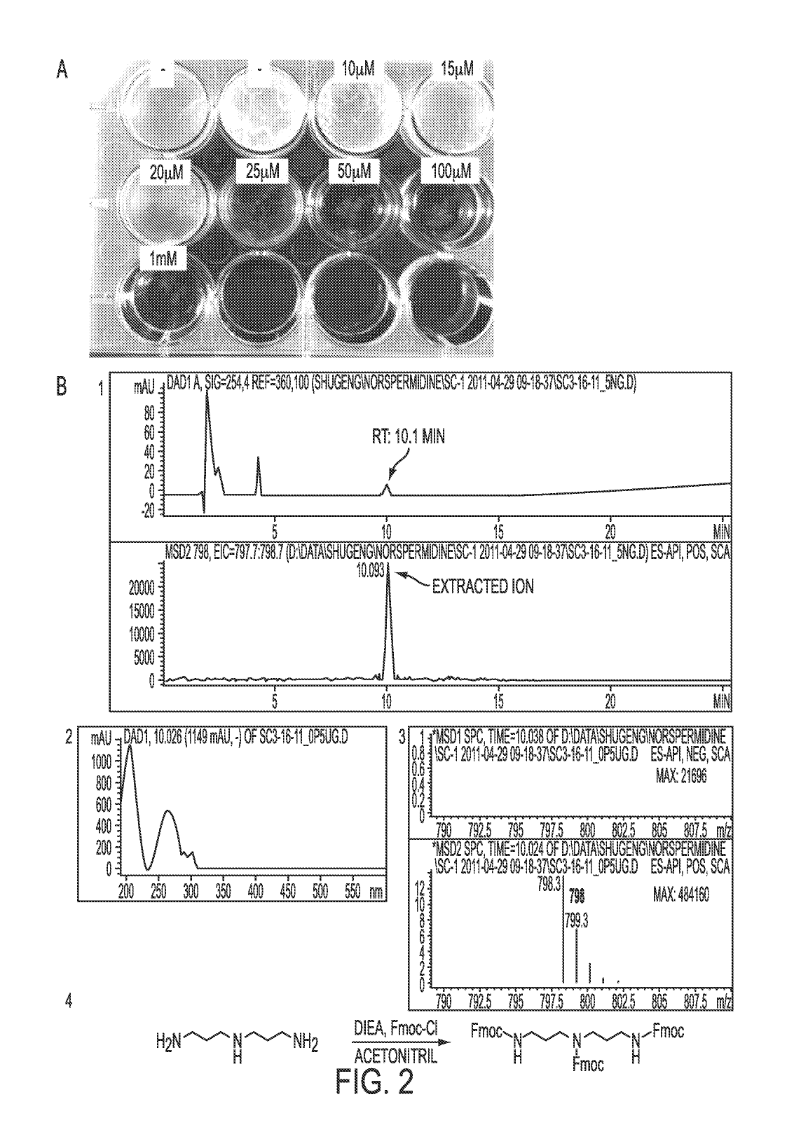

In one or more embodiments, polyamines can inhibit biofilm formation in cell populations, and in particular, in biofilm-forming bacteria. By way of example, polyamines such as norspermidine and norspermine significantly retard the formation of biofilm in bacterial colonies such as Staphylococcus aureus, Bacillus subtilis, and Pseudomonas aeruginosa. See, e.g., FIGS. 1, 2, 7 and 9, and Examples 1, 2, 7, and 9. Polyamines have been demonstrated to reduce biofilm-forming activity by measuring the OD600 of cells that adhere to the surface as a measure of biofilm formation.

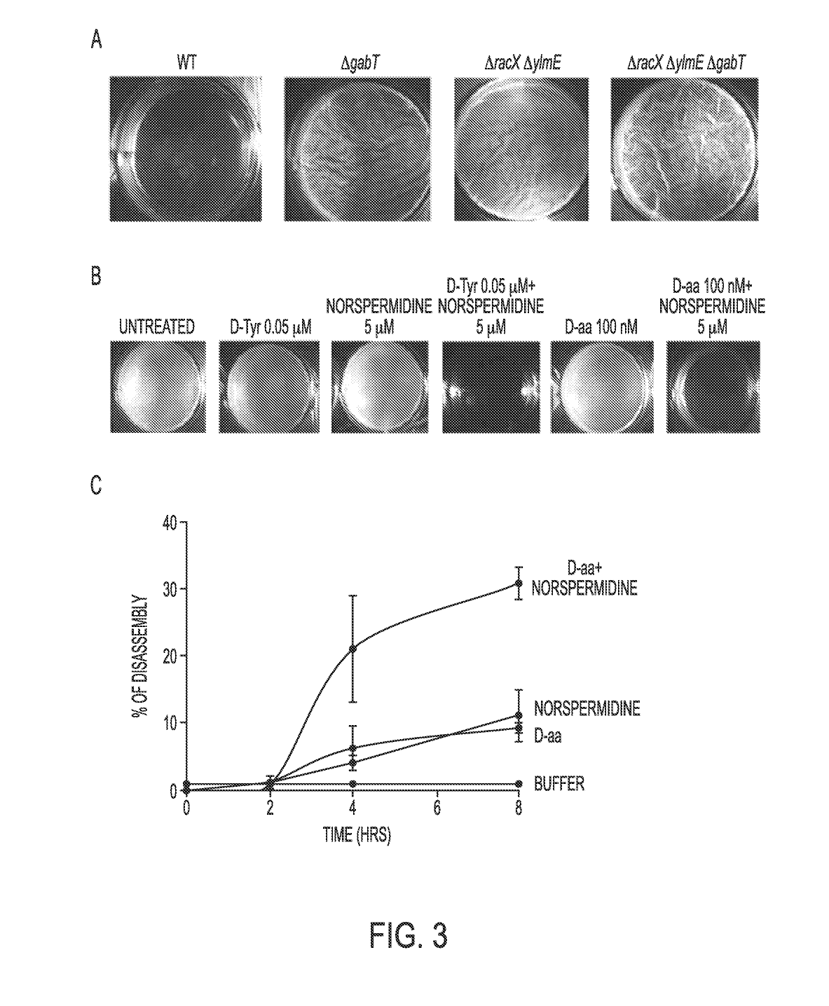

In one or more embodiments, polyamines can disrupt established biofilms. Even after bacteria have established a biofilm, contact of the biofilm with a solution containing a polyamine results in the disruption and disassembly of the pellicle. By way of example, polyamines such as norspermine can disrupt pellicles formed by bacterial colonies such as Bacillus subtilis. See, e.g., FIG. 3 and Example 3. Polyamines have been demonstrated to reduce biofilm-forming activity by measuring the OD600 of cells in free medium and compared to the OD600 in the residual pellicle.

In one aspect of this disclosure, a biofilm-related disorder present in a subject in need thereof is treated by administering to the subject a composition having an effective amount of a polyamine, or a pharmaceutically acceptable salt, or derivative thereof, thereby treating the biofilm-related disorder. A polyamine can be administered at a concentration of 0.1 nM to 100 μM, e.g., 1 nM to 10 μM, 5 nM to 5 μM, or 10 nM to 1 μM. In other embodiments, a polyamine can be administered at a concentration of about 0.1 nM to about 100 μM, e.g., about 1 nM to about 10 μM, about 5 nM to about 5 μM, or about 10 nM to about 1 μM.

Exemplary polyamines found to be particularly effective in inhibiting or treating biofilm formation include norspermidine, norspermine, 1,5,9-triazacyclododecane, and other compounds in Table 1. Norspermidine, norspermine, or 1,5,9-triazacyclododecane can be used, for example, at concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

### Polyamines and Amino Acids

It has been surprisingly discovered that a polyamine and a D-amino acid can act synergistically to inhibit biofilm formation or trigger biofilm disassembly. It has been discovered that polyamines and D-amino acids inhibit biofilm formation and trigger biofilm disassembly by different mechanisms. Thus, in one or more embodiments, a polyamine can be co administered with an amino acid, and in particular with a D-amino acid, to inhibit biofilm formation or trigger biofilm disassembly. The different mechanisms by which the polyamines and D-amino acids work result in synergism between the polyamine and D-amino acid and, in some embodiments, lower amounts of polyamines and D-amino acids are used to inhibit biofilm formation and/or trigger biofilm disassembly.

Standard amino acids can exist in either of two optical isomers, called L- or D-amino acids, which are mirror images of each other. While L-amino acids represent the vast majority of amino acids found in proteins, D-amino acids are components of the peptidoglycan cell walls of bacteria. The D-amino acids described herein are capable of penetrating biofilms on living and non-living surfaces, of preventing the adhesion of bacteria to surfaces and any further build-up of the biofilm, of detaching such biofilm and/or inhibiting the further growth of the biofilm-forming micro-organisms in the biological matrix, or of killing such micro-organisms. D-amino acids are known in the art and can be prepared using known techniques. Exemplary methods include, e.g., those described in U.S. Publ. No. 20090203091. D-amino acids are also commercially available (e.g., from Sigma Chemicals, St. Louis, Mo.).

Any D-amino acid can be used in the methods described herein, including without limitation D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, or D-tyrosine. A D-amino acid can be used alone or in combination with other D-amino acids. In exemplary methods, 2, 3, 4, 5, 6, or more D-amino acids are used in combination. Preferably, D-tyrosine, D-leucine, D-methionine, or D-tryptophan, either alone or in combination, are used in the methods described herein. In other preferred embodiments, D-tyrosine, D-proline and D-phenylalanine, either alone or in combination, are used in the methods described herein.

A D-amino acid combined with a polyamine can be administered at a concentration of 0.1 nM to 100 μM, e.g., 1 nM to 10 μM, 5 nM to 5 μM, or 10 nM to 1 μM. In other embodiments, a D-amino acid can be administered at a concentration of about 0.1 nM to about 100 μM, e.g., about 1 nM to about 10 μM, about 5 nM to about 5 μM, or about 10 nM to about 1 μM.

An exemplary D-amino acid found to be particularly effective in inhibiting or treating biofilm formation when combined with a polyamine includes D-tyrosine. In some embodiments, D-tyrosine can be used, for example, as concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

In other embodiments, D-tyrosine is used in combination with one or more of D-proline and D-phenylalanine. In some embodiments, D-tyrosine is used in combination with one or more of D-leucine, D-tryptophan, and D-methionine. The combinations of D-tyrosine with one or more of D-proline, D-phenylalanine, D-leucine, D-tryptophan, and D-methionine can be synergistic and can be effective in inhibiting or treating biofilm formation at total D-amino acid concentrations of 10 μM or less, e.g., about 1 nM to about 10 μM, about 5 nM to about 5 μM, or about 10 nM to about 1 μM, or at a concentration of 0.1 nM to 100 μM, e.g., 1 nM to 10 μM, 5 nM to 5 μM, or 10 nM to 1 μM.

In some embodiments, the combinations of polyamines and D-amino acids are equimolar. In some embodiments, the combinations of D-amino acids are equimolar. In other embodiments, the combinations of D-amino acids are not in equimolar amounts.

In some embodiments, the composition is essentially free of L-amino acids. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of L-amino acids. In other embodiments, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001% of L-amino acids. In preferred embodiments, the percentage of L-amino acid is relative to the corresponding D-amino acid. By way of example, a racemic mixture of L-amino acid and D-amino acid contains 50% L-amino acid.

In some embodiments, the composition is essentially free of detergent. For example, the composition comprises, less than about 30 wt %, less than about 20 wt %, less than about 10 wt %, less than about 5 wt %, less than about 1 wt %, less than about 0.5 wt %, less than about 0.25 wt %, less than about 0.1 wt %, less than about 0.05 wt %, less than about 0.025 wt %, less than about 0.01 wt %, less than about 0.005 wt %, less than about 0.0025 wt %, less than about 0.001 wt %, or less, of a detergent. In other embodiments, the composition comprises, relative to the overall composition, less than about 30 wt %, less than 20 wt %, less than 10 wt %, less than 5 wt %, less than 1 wt %, less than 0.5 wt %, less than 0.25 wt %, less than 0.1 wt %, less than 0.05 wt %, less than 0.025 wt %, less than 0.01 wt %, less than 0.005 wt %, less than 0.0025 wt %, less than 0.001 wt % of a detergent. Many times in formulations containing detergents, e.g., surfactants, the surfactant will interact with the active agent, which could greatly affect the agent's efficacy. In some embodiments, it can be necessary to screen agents effectiveness relative to anionic surfactants, cationic surfactants, non-ionic surfactants and zwitter ionic surfactants as a screening to determine if the presence of the surfactant type alters the efficacy. Reducing or eliminating detergents, can increase the efficacy of the compositions and/or reduce formulation complications.

In other embodiments, the composition is essentially free of both detergent and L-amino acids.

### Biofilms

Most bacteria can form complex, matrix-containing multicellular communities known as biofilms (O'Toole et al., Annu Rev. Microbiol. 54:49 (2000); Lopez et al., FEMS Microbiol. Rev. 33:152 (2009); Karatan et al., Microbiol. Mol. Biol. Rev. 73:310 (2009)). Biofilm-associated bacteria are protected from environmental insults, such as antibiotics (Bryers, Biotechnol. Bioeng. 100:1 (2008)). However, as biofilms age, nutrients become limiting, waste products accumulate, and it is advantageous for the biofilm-associated bacteria to return to a planktonic existence (Karatan et al., Microbiol. Mol. Biol. Rev. 73:310 (2009)). Thus, biofilms have a finite lifetime, characterized by eventual disassembly.

Gram-negative bacteria, Gram-positive bacteria, and mycobacteria, in addition to other unicellular organisms, can produce biofilms. Bacterial biofilms are surface-attached communities of cells that are encased within an extracellular polysaccharide matrix produced by the colonizing cells. Biofilm development occurs by a series of programmed steps, which include initial attachment to a surface, formation of three-dimensional microcolonies, and the subsequent development of a mature biofilm. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins). A biofilm may include an integrated community of two or more bacteria species (polymicrobic biofilms), or predominantly one specific bacterium.

The biofilm can allow bacteria to exist in a dormant state for a certain amount of time until suitable growth conditions arise thus offering the microorganism a selective advantage to ensure its survival. However, this selection can pose serious threats to human health in that biofilms have been observed to be involved in about 65% of human bacterial infections (Smith, Adv. Drug Deliv. Rev. 57:1539-1550 (2005); Hall-Stoodley et al., Nat. Rev. Microbiol. 2:95-108 (2004)).

As described herein, biofilms can also affect a wide variety of biological, medical, and processing operations.

### Biofilm-Forming Bacteria

The methods described herein can be used to prevent or delay the formation of, and/or treat, biofilms. In exemplary methods, the biofilms are formed by biofilm-forming bacteria. The bacteria can be a gram negative bacterial species or a gram positive bacterial species. Nonlimiting examples of such bacteria include a member of the genus Actinobacillus (such as Actinobacillus actinomycetemcomitans), a member of the genus Acinetobacter (such as Acinetobacter baumannii), a member of the genus Aeromonas, a member of the genus Bordetella (such as Bordetella pertussis, Bordetella bronchiseptica, or Bordetella parapertussis), a member of the genus Brevibacillus, a member of the genus Brucella, a member of the genus Bacteroides (such as Bacteroides fragilis), a member of the genus Burkholderia (such as Burkholderia cepacia or Burkholderia pseudomallei), a member of the genus Borelia (such as Borelia burgdorferi), a member of the genus Bacillus (such as Bacillus anthracis or Bacillus subtilis), a member of the genus Campylobacter (such as Campylobacter jejuni), a member of the genus Capnocytophaga, a member of the genus Cardiobacterium (such as Cardiobacterium hominis), a member of the genus Citrobacter, a member of the genus Clostridium (such as Clostridium tetani or Clostridium difficile), a member of the genus Chlamydia (such as Chlamydia trachomatis, Chlamydia pneumoniae, or Chlamydia psiffaci), a member of the genus Eikenella (such as Eikenella corrodens), a member of the genus Enterobacter, a member of the genus Escherichia (such as Escherichia coli), a member of the genus Francisella (such as Francisella tularensis), a member of the genus Fusobacterium, a member of the genus Flavobacterium, a member of the genus Haemophilus (such as Haemophilus ducreyi or Haemophilus influenzae), a member of the genus Helicobacter (such as Helicobacter pylori), a member of the genus Kingella (such as Kingella kingae), a member of the genus Klebsiella (such as Klebsiella pneumoniae), a member of the genus Legionella (such as Legionella pneumophila), a member of the genus Listeria (such as Listeria monocytogenes), a member of the genus Leptospirae, a member of the genus Moraxella (such as Moraxella catarrhalis), a member of the genus Morganella, a member of the genus Mycoplasma (such as Mycoplasma hominis or Mycoplasma pneumoniae), a member of the genus Mycobacterium (such as Mycobacterium tuberculosis or Mycobacterium leprae), a member of the genus Neisseria (such as Neisseria gonorrhoeae or Neisseria meningitidis), a member of the genus Pasteurella (such as Pasteurella multocida), a member of the genus Proteus (such as Proteus vulgaris or Proteus mirablis), a member of the genus Prevotella, a member of the genus Plesiomonas (such as Plesiomonas shigelloides), a member of the genus Pseudomonas (such as Pseudomonas aeruginosa), a member of the genus Providencia, a member of the genus Rickettsia (such as Rickettsia rickettsii or Rickettsia typhi), a member of the genus Stenotrophomonas (such as Stenotrophomonas maltophila), a member of the genus Staphylococcus (such as Staphylococcus aureus or Staphylococcus epidermidis), a member of the genus Streptococcus (such as Streptococcus viridans, Streptococcus pyogenes (group A), Streptococcus agalactiae (group B), Streptococcus bovis, or Streptococcus pneumoniae), a member of the genus Streptomyces (such as Streptomyces hygroscopicus), a member of the genus Salmonella (such as Salmonella enteriditis, Salmonella typhi, or Salmonella typhimurium), a member of the genus Serratia (such as Serratia marcescens), a member of the genus Shigella, a member of the genus Spirillum (such as Spirillum minus), a member of the genus Treponema (such as Treponema pallidum), a member of the genus Veillonella, a member of the genus Vibrio (such as Vibrio cholerae, Vibrio parahaemolyticus, or Vibrio vulnificus), a member of the genus Yersinia (such as Yersinia enterocolitica, Yersinia pestis, or Yersinia pseudotuberculosis), and a member of the genus Xanthomonas (such as Xanthomonas maltophilia).

Specifically, Bacillus subtilis forms architecturally complex communities on semi-solid surfaces and thick pellicles at the air/liquid interface of standing cultures (Lopez et al., FEMS Microbiol. Rev. 33:152 (2009); Aguilar et al., Curr. Opin. Microbiol. 10:638 (2007); Vlamakis et al., Genes Dev. 22:945 (2008); Branda et al., Proc. Natl. Acad. Sci. USA 98:11621 (2001)). B. subtilis biofilms consist of long chains of cells held together by an extracellular matrix consisting of an exopolysaccharide and amyloid fibers composed of the protein TasA (Branda et al., Proc. Natl. Acad. Sci. USA 98:11621 (2001); Branda et al., Mol. Microbiol. 59:1229 (2006); Romero et al., Proc. Natl. Acad. Sci. USA (2010, in press)). The exopolysaccharide is produced by enzymes encoded by the epsA-O operon (“eps operon”) and the TasA protein is encoded by the promoter-distal gene of the yqxM-sipW-tasA operon (“yqxM operon”) (Chu et al., Mol. Microbiol. 59:1216 (2006)).

Biofilm-producing bacteria, e.g., a species described herein, can be found in a live subject, in vitro, or on a surface, as described herein.

### Applications/Formulations

In instances where a polyamine, or a combination of a polyamine and a D-amino acid, is to be administered to a subject, the polyamines and D-amino acids described herein can be incorporated into pharmaceutical compositions. The polyamines and D-amino acids can be incorporated into pharmaceutical compositions as pharmaceutically acceptable salts or derivatives. Such compositions typically include a polyamine, or a polyamine and a D-amino acid, and a pharmaceutically acceptable carrier. As used herein, a “pharmaceutically acceptable carrier” means a carrier that can be administered to a subject together with a polyamine or with a polyamine and a D-amino acid described herein, which does not destroy the pharmacological activity thereof. Pharmaceutically acceptable carriers include, e.g., solvents, binders, dispersion media, coatings, preservatives, colorants, isotonic and absorption delaying agents, and the like, compatible with pharmaceutical administration. Supplementary active compounds can also be incorporated into the compositions.

The term “pharmaceutically acceptable salts” includes, but is not limited to, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

The D-amino acids may also be in the form of esters or derivatives. Examples of suitable esters include formates, acetates, propionates, butyrates, isobutyrates, pentanoates, crotonates, and benzoates. Some pharmaceutically acceptable derivatives include a chemical group which increases aqueous solubility.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid (PLGA), polycaprolactone, polyvalerolactone, poly(anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In other instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and/or combinations thereof.

A pharmaceutical composition containing a polyamine, or a combination of a polyamine and a D-amino acid, can be formulated to be compatible with its intended route of administration as known by those of ordinary skill in the art. Nonlimiting examples of routes of administration include parenteral, e.g., intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition can be sterile and can be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21st edition, Lippincott Williams & Wilkins, Gennaro, ed. (2006)).

Sterile injectable solutions can be prepared by incorporating a polyamine, or combination of a polyamine and a D-amino acid, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, a polyamine, or a combination of a polyamine and a D-amino acid, can be incorporated with excipients and used in the form of tablets, pills, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, a polyamine, or a combination of a polyamine and a D-amino acid, can be delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays or suppositories. For transdermal administration, the active compounds are formulated into, e.g., ointments, salves, gels, or creams as generally known in the art.

For treatment of acute or chronic wounds, a polyamine, or a combination of a polyamine and a D-amino acid, can be formulated as a dressing, a wash solution, gel, or a synthetic tissue.

A biofilm can form on an oral surface (such as teeth, tongue, back of throat, and the like). These biofilms can be associated with day-to-day bacterial activity of natural flora located in such environments, but can also be associated with oral-related disease(s), such as periodontal disease (for example, gingivitis or periodontitis), breath malodor, or dental caries. By example, periodontitis, a common form of periodontal disease, is believed to be caused by a small group of Gram-negative bacteria present on the tooth root surfaces as biofilms, in particular, Porphyromonas gingivalis, Bacteroides forsythus and Actinobacillus actinomycetemcomitans, with the latter found mostly in cases of juvenile periodontitis. Other bacteria which may be involved in periodontal disease include T. denticola, T. socranskii, F. nucleatum, and P. intermedia, L. acidophilus, L. casei, A. viscosus, S. sobrinus, S sanguis, S. viridans, and S. mutans. Application of a polyamine, or a combination of a polyamine and a D-amino acid, onto such oral surfaces can inhibit or prevent bacterial biofilm formation. Generally, application onto such oral surfaces will be via a product which, in the ordinary course of usage, is not intentionally swallowed for purposes of systemic administration but is rather retained in the oral cavity for a time sufficient to contact substantially all of the dental surfaces and/or oral tissues. The polyamine, or combination of a polyamine and D-amino acid, for use on oral surfaces can be formulated as a gum, paste (such as toothpaste), which can then be directly applied to the biofilm of such a surface in a subject. The paste formulation can further comprise an abrasive. A polyamine, or combination of a polyamine and D-amino acid, can also exist as a gel formulation or in liquid formulation. For example, the polyamine, or combination of a polyamine and D-amino acid, can be formulated as a mouthwash that can directly come into contact with the biofilm on the oral surface of a subject. Additionally, a polyamine, or combination of a polyamine and D-amino acid, can be formulated as a polymer film or platelet (e.g., as a slow-release formulation) for treating or preventing oral conditions. In one embodiment, the polyamine, or combination of a polyamine and D-amino acid, of the present invention may be used for adjunctive antimicrobial therapy for periodontitis and applied directly to a tooth or between teeth in the form of a chip. The oral care compositions of the present invention may be in various forms including therapeutic rinses, especially mouth rinses; dentifrices such as toothpastes, tooth gels, and tooth powders; non-abrasive gels; mouth sprays; mousse; foams; chewing gums, lozenges and breath mints; drinking water additives; dental solutions and irrigation fluids; and dental implements such as dental floss and tape. The dental implement can be impregnated fibers including dental floss or tape, chips, strips, films and polymer fibers.

For example, an oral composition can contain from about 0.01% to about 15% by weight, e.g., 0.01% to 15% by weight, based on the total weight of the composition, of one or more polyamine, or combination of a polyamine and D-amino acid, and orally tolerable adjuvants. One nonlimiting example of an oral composition includes 10% by weight sorbitol, 10% by weight glycerol, 15% by weight ethanol, 15% by weight propylene glycol, 0.5% by weight sodium lauryl sulfate, 0.25% by weight sodium methylcocyl taurate, 0.25% by weight polyoxypropylene/polyoxyethylene block copolymer, 0.10% by weight peppermint flavouring, 0.1 to 0.5% by weight of one or more polyamine, or combination of a polyamine and D-amino acid, and 48.6% by weight water.

An oral composition can be, for example, in the form of a gel, a paste, a cream or an aqueous preparation (mouthwash). The oral composition can also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name OLAFLUOR. Oral compositions can further comprise compounds known in the art to be “orally acceptable carriers,” which as used herein means conventional additives in oral care compositions including but not limited to fluoride ion sources, anti-calculus or anti-tartar agents, buffers, abrasives such as silica, bleaching agents such as peroxide sources, alkali metal bicarbonate salts, thickening materials, humectants, water, surfactants, titanium dioxide, flavor system, sweetening agents, xylitol, coloring agents, and mixtures thereof. Such materials are well known in the art and are readily chosen by one skilled in the art based on the physical, aesthetic and performance properties desired for the compositions being prepared. These carriers may be included at levels typically from about 50% to about 99%, preferably from about 70% to about 98%, and more preferably from about 90% to about 95%, by weight of the oral composition. The choice of a carrier to be used is basically determined by the way the composition is to be introduced into the oral cavity. In one preferred embodiment, the oral compositions are in the form of dentifrices, such as toothpastes, tooth gels and tooth powders. Components of such toothpaste and tooth gels generally include one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders contain substantially all non-liquid components. Other preferred oral care compositions are liquid products, including mouthwashes or rinses, mouth sprays, dental solutions and irrigation fluids. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

The pharmaceutical compositions containing a polyamine, or combination of a polyamine and D-amino acid, can also be prepared in the form of suppositories (e.g., with conventional suppository bases such as cocoa butter and other glycerides) or retention enemas for rectal delivery.

In some embodiments, the composition is essentially free of detergent. In some instances, a detergent can contribute to the toxicity of a composition. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of a detergent, e.g., less than 30%, less than 20%, less than 10%, less than 5%, less than 1%, less than 0.5%, less than 0.25%, less than about 0.1%, less than 0.05%, less than 0.025%, less than 0.01%, less than 0.005%, less than about 0.0025%, less than 0.001%, of a detergent.

Some pharmaceutical compositions can be prepared with a carrier that protects the polyamine, or combination of a polyamine and D-amino acid, against rapid elimination from the body, such as a controlled release formulation, including implants and microencapsulated delivery systems (as described, e.g., in Tan et al., Pharm. Res. 24:2297-2308, 2007). Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially (e.g., from Alza Corp., Mountain View, Calif.). Liposomal suspensions (including liposomes targeted to particular cells with monoclonal antibodies to cell surface antigens) can also be used as pharmaceutically acceptable carriers. These can be prepared according to methods known to those skilled in the art, e.g., as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects can be used, care should be taken to design a delivery system that targets such compounds to the site of affected tissue in order to minimize potential damage to normal cells and, thereby, reduce side effects.

The data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art (see, e.g., Remington: The Science and Practice of Pharmacy, 21st edition, Lippincott Williams & Wilkins, Gennaro, ed. (2006)).

In some instances, about 0.0005 μM polyamine to about 50 μM polyamine is administered, e.g., about 0.001 μM polyamine to about 25 μM polyamine, about 0.002 μM polyamine to about 10 μM polyamine, about 0.003 μM polyamine to about 5 μM polyamine, about 0.004 μM polyamine to about 1 μM polyamine, about 0.005 μM polyamine to about 0.5 μM polyamine, about 0.01 μM polyamine to about 0.1 μM polyamine, or about 0.02 μM polyamine to about 0.1 μM polyamine, e.g., 0.0005 μM polyamine to 50 μM polyamine is administered, 0.001 μM polyamine to 25 μM polyamine, 0.002 μM polyamine to 10 μM polyamine, 0.003 μM polyamine to 5 μM polyamine, 0.004 μM polyamine to 1 μM polyamine, 0.005 μM polyamine to 0.5 μM polyamine, 0.01 μM polyamine to 0.1 μM polyamine, or 0.02 μM polyamine to 0.1 μM polyamine.

When combined with a polyamine, about 0.0005 μM D-amino acid to about 50 μM D-amino acid is administered, e.g., about 0.001 μM D-amino acid to about 25 μM D-amino acid, about 0.002 μM D-amino acid to about 10 μM D-amino acid, about 0.003 μM D-amino acid to about 5 μM D-amino acid, about 0.004 μM D-amino acid to about 1 μM D-amino acid, about 0.005 μM D-amino acid to about 0.5 μM D-amino acid, about 0.01 μM D-amino acid to about 0.1 μM D-amino acid, or about 0.02 μM D-amino acid to about 0.1 μM D-amino acid, e.g., 0.0005 μM D-amino acid to 50 μM D-amino acid is administered, 0.001 μM D-amino acid to 25 μM D-amino acid, 0.002 μM D-amino acid to 10 μM D-amino acid, 0.003 μM D-amino acid to 5 μM D-amino acid, 0.004 μM D-amino acid to 1 μM D-amino acid, 0.005 μM D-amino acid to 0.5 μM D-amino acid, 0.01 μM D-amino acid to 0.1 μM D-amino acid, or 0.02 μM D-amino acid to 0.1 μM D-amino acid. Preferably, a D-amino acid is administered at nanomolar concentrations, e.g., at about 5 nM, at about 10 nM, at about 15 nM, at about 20 nM, at about 25 nM, at about 30 nM, at about 50 nM, or more, or preferably at 5 nM, at 10 nM, at 15 nM, at 20 nM, at 25 nM, at 30 nM, or at 50 Nm.

In other instances, a therapeutically effective amount or dosage of a polyamine, or combination of a polyamine and D-amino acid, can range from about 0.001 mg/kg body weight to about 100 mg/kg body weight, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight, or preferably 0.001 mg/kg body weight to 100 mg/kg body weight, e.g., from 0.01 mg/kg body weight to 50 mg/kg body weight, from 0.025 mg/kg body weight to 25 mg/kg body weight, from 0.1 mg/kg body weight to 20 mg/kg body weight, from 0.25 mg/kg body weight to 20 mg/kg body weight, from 0.5 mg/kg body weight to 20 mg/kg body weight, from 0.5 mg/kg body weight to 10 mg/kg body weight, from 1 mg/kg body weight to 10 mg/kg body weight, or 5 mg/kg body weight.

A physician will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polyamine, or combination of a polyamine and D-amino acid, can include a single treatment or a series of treatments. In one example, a subject is treated with a polyamine, or combination of a polyamine and D-amino acid, in the range of between about 0.06 mg to about 120 mg, one time per week for between about 1 to 10 weeks, alternatively between 2 to 8 weeks, between about 3 to 7 weeks, or for about 4, 5, or 6 weeks, or preferably between 0.06 mg to 120 mg, one time per week for between 1 to 10 weeks, alternatively between 2 to 8 weeks, between 3 to 7 weeks, or for 4, 5, or 6 weeks. It will also be appreciated that the effective dosage of a polyamine, or combination of a polyamine and D-amino acid, used for treatment may increase or decrease over the course of a particular treatment.

The pharmaceutical compositions can be included in a container, pack, or dispenser together with instructions for administration. A person of ordinary skill in the art will appreciate that the pharmaceutical compositions described herein can be formulated as single-dose vials.

Treatment of a subject with a therapeutically effective amount of a polyamine-, or combination of a polyamine and D-amino acid-, containing pharmaceutical composition described herein can be a single treatment, continuous treatment, or a series of treatments divided into multiple doses. The treatment can include a single administration, continuous administration, or periodic administration over one or more years. Chronic, long-term administration can be indicated in some cases. Generally, each formulation is administered in an amount sufficient to suppress or reduce or eliminate a deleterious effect or a symptom of a biofilm-related disorder or condition described herein.

Polyamines, or combinations of polyamines and D-amino acids, are suitable as antibiofilm active substances in personal care preparations, for example shampoos, bath additives, hair care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, oils or powders. Propionibacterium acnes, which is the predominant microorganism occurring in acne, may reside in biofilms. Thus, polyamines, or combinations of polyamines and D-amino acids, are particularly suitable for personal care compositions for use in controlling acne. The invention accordingly relates also to personal care preparations comprising one or more polyamine, or a combination of a polyamine and D-amino acid, described herein and cosmetically tolerable carriers or adjuvants.

The polyamines, or combinations of polyamines and D-amino acids, described herein are also suitable for imparting antibiofilm properties to a range of formulations used in personal care. Personal care preparations can contain from about 0.01% to about 15% by weight, for example, from about 0.1% to about 10% by weight, or 0.01% to 15% by weight, for example, from 0.1% to 10% by weight, based on the total weight of the preparation, of one or more polyamine, or combination of a polyamine and D-amino acid, and cosmetically tolerable adjuvants. Depending on the form of the personal care preparation, such preparation can include, in addition to one or more polyamines, or combinations of polyamines and D-amino acids, further constituents, for example sequestering agents, colourings, perfume oils, thickening or solidifying agents (consistency regulators), emollients, UV-absorbers, skin protective agents, antioxidants, additives that improve the mechanical properties, such as dicarboxylic acids and/or aluminium, zinc, calcium or magnesium salts of C₁₄-C₂₂ fatty acids, and, optionally, preservatives.

In one embodiment, the anti-acne composition comprising a polyamine, or a combination of a polyamine and D-amino acid, can further comprise at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides. The present invention provides a method for controlling acne, comprising administering to a subject afflicted with acne an effective amount of an anti-acne composition comprising one or more polyamines, or combinations of polyamines and D-amino acids, wherein the amount of the polyamine, or combination of a polyamine and D-amino acid, in the anti-acne composition is sufficient to prevent, reduce, inhibit or remove a biofilm.

Personal care preparations can be in the form of a water-in-oil or oil-in-water emulsion, an alcoholic or alcohol-containing formulation, a vesicular dispersion of an ionic or non-ionic ampiphilic lipid, a gel, a solid stick or an aerosol formulation. As a water-in-oil or oil-in-water emulsion, the cosmetically tolerable adjuvant contains preferably from about 5% to about 50% of an oil phase, from about 5% to about 20% of an emulsifier and from about 30% to 90% water, or 5% to 50% of an oil phase, from 5% to 20% of an emulsifier and from 30% to 90% water. The oil phase can comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations described herein are used in various fields. Such preparations include, without limitation, for example:
skin-care preparations, e.g. skin-washing and cleansing preparations in the form of tablet-form or liquid soaps, synthetic detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eye shadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams or oils, sun-blocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
dental care, denture-care and mouth-care preparations, e.g. toothpastes, gel toothpastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

The following represent nonlimiting examples of various formulations that can be prepared containing one or more polyamines, or combinations of polyamines and D-amino acids. A wide variety of similar formulations are known in the art into which one or more polyamines, or combinations of polyamines and D-amino acids, can readily be incorporated at various concentrations.

An exemplary soap has, for example, the following composition: 0.01 to 5% by weight of one or more polyamines, or combinations of polyamines and D-amino acids, 0.3 to 1% by weight titanium dioxide, 1 to 10% by weight stearic acid, soap base ad 100%, e.g. a sodium salt of tallow fatty acid or coconut fatty acid, or glycerol.

An exemplary shampoo has, for example, the following composition: 0.01 to 5% by weight of one or more polyamines, or combination of a polyamine and D-amino acid, 12.0% by weight sodium laureth-2-sulfate, 4.0% by weight cocamidopropyl betaine, 3.0% by weight NaCl and water ad 100%.

An exemplary deodorant has, for example, the following composition: 0.01 to 5% by weight of one or more polyamines, or combination of a polyamine and D-amino acid, 60% by weight ethanol, 0.3% by weight perfume oil, and water ad 100%.

In some instances, a pharmaceutical composition comprising a polyamine, or combination of a polyamine and D-amino acid, is administered to prevent or reduce biofilm formation on a biologically relevant surface or substrate. These surfaces include, but are not limited to, an epithelial or mucosal surface of the respiratory tract, lungs, the oral cavity, the alimentary and vaginal tracts, in the ear or the surface of the eye, and the urinary tract. For example, a biofilm can affect the surface of a lung (such as the lung of a subject with pneumonia, cystic fibrosis, or COPD), such as epithelial cells of the lung.

In certain embodiments, the surface is a biologically relevant surface is a surface that is likely to contact a biological fluid, e.g., a liquid component of a subject such as blood, serum, sputum, lacrimal secretions, semen, urine, vaginal secretions, and tissue samples and the like. The biologically relevant surface can be a component of a medical device, instrument, or implant. Nonlimiting examples include clamps, forceps, scissors, skin hooks, tubing (such as endotracheal or gastrointestinal tubes), needles, retractors, scalers, drills, chisels, rasps, saws, catheters including indwelling catheter (such as urinary catheters, vascular catheters, peritoneal dialysis catheter, central venous catheters), catheter components (such as needles, Leur-Lok connectors, needleless connectors), orthopedic devices, artificial heart valves, prosthetic joints, voice prostheses, stents, shunts, pacemakers, surgical pins, respirators, ventilators, and endoscopes. The present invention is particularly well-suited to substantially reduce the risk of biofilm accumulation on the surfaces of a medical device adapted for prolonged term implantation, wherein the medical device is intended to remain implanted for a relatively long period of from about 30 days to about 12 months or longer, and the resultant likelihood of premature failure of the device due to encrustation and occlusion by such biofilm. However, such encrustation may occur on medical devices after shorter periods of time, such as 30 days or less, as well, which would also be understood to be devices for prolonged term implantation. For example, in certain embodiments, a medical device utilized for a prolonged period of time may implanted for a period longer than 24 hours, such as a week.

In certain instances, a subject can be administered a polyamine, or combination of a polyamine and D-amino acid, prior to, during, or after implantation/insertion of a medical device, catheter, stent, prosthesis, and the like, or application of a wound dressing. In some instances, the wound dressing includes an antimicrobial, such as silver. Treatment before or after implantation can take place immediately before or after the implantation or several hours before or after implantation, or at a time or times that the skilled physician deems appropriate.

A polyamine, or combination of a polyamine and D-amino acid, can be applied to a surface by any known means, such as by covering, coating, contacting, associating with, filling, or loading the surface with a therapeutic amount of a polyamine, or combination of a polyamine and D-amino acid. In specific examples, a polyamine, or combination of a polyamine and D-amino acid, is directly affixing to a surface by either spraying the surface with a polymer/polyamine, or a polymer/combination of a polyamine and D-amino acid, film, by dipping the surface into a polymer/polyamine solution, or a polymer/combination of a polyamine and D-amino acid solution, or by other covalent or noncovalent means. In other instances, the surface is coated with a substance (such as a hydrogel) that absorbs the polyamine, or combination of a polyamine and D-amino acid.

The composition can be a coating or a film. When applied as a part of a film or coating, one or more polyamine, or combination of a polyamine and D-amino acid, described herein can be part of a composition which also comprises a binder. The binder may be any polymer or oligomer compatible with the present antibiofilms. The binder may be in the form of a polymer or oligomer prior to preparation of the antibiofilm composition, or may form by polymerization during or after preparation, including after application to the substrate. In certain applications, such as certain coating applications, it will be desirable to crosslink the oligomer or polymer of the antibiofilm composition after application. The term “binder” as used herein includes materials such as glycols, oils, waxes and surfactants commercially used in the pharmaceutical and personal care industries. It is preferred that materials that are Generally Regarded as Safe (G.R.A.S.) be used.

When the composition is a thermoplastic film which is applied to a surface, for example, by the use of an adhesive or by melt applications including calendaring and co-extrusion, the binder is the thermoplastic polymer matrix used to prepare the film. When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer. Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g. from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention. Alkyd resins, polyesters, polyurethanes, epoxy resins, silicone containing polymers, polyacrylates, polyacrylamides, fluorinated polymers and polymers of vinyl acetate, vinyl alcohol and vinyl amine are non-limiting examples of common coating binders useful in the present invention. Other known coating binders are part of the present disclosure.

Coatings can be crosslinked with, for example, melamine resins, urea resins, isocyanates, isocyanurates, polyisocyanates, epoxy resins, anhydrides, poly acids and amines, with or without accelerators. The compositions described herein can be, for example, a coating applied to a surface which is exposed to conditions favorable for bioaccumulation. The presence of one or more polyamines, or combinations of polyamines and D-amino acids, described herein in said coating can prevent the adherence of organisms to the surface.

The coating may be solvent borne or aqueous. Aqueous coatings are typically considered more environmentally friendly. In some examples, the coating can be an aqueous dispersion of one or more polyamines, or combinations of polyamines and D-amino acids, described herein and a binder or a water based coating or paint. For example, the coating can comprise an aqueous dispersion of one or more polyamines, or combinations of polyamines and D-amino acids, and an acrylic, methacrylic or acrylamide polymers or co-polymers or a poly[-alkoxyalkanoate-co-3-hydroxyalkenoate] polyester.

In some instances, the coating composition can be applied to a surface by any conventional means including spin coating, dip coating, spray coating, draw down, or by brush, roller or other applicator. A drying or curing period can be performed.

Coating or film thickness can vary depending on the application and can readily be determined by one skilled in the art after limited testing.

In some instances, a composition described herein can be in the form of a protective laminate film. Such a film can comprise thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, fluorinated polymers, silicon containing and carbamate polymers. The polymers can also be blends and copolymers of the preceding chemistries.

When a composition described herein is a preformed film, it can be applied to a surface by, for example, the use of an adhesive, or co-extruded onto the surface. It can also be mechanically affixed via fasteners which may require the use of a sealant or caulk wherein the esters of the instant invention may also be advantageously employed. A plastic film can also be applied with heat which includes calendaring, melt applications and shrink wrapping.

Given the wide array of applications for the polyamines, or combinations of polyamines and D-amino acids, described herein, a polyamine-containing composition, or a composition containing a combination of a polyamine and D-amino acid, can include other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

Medical devices prepared from plastic can incorporate a polyamine, or combination of a polyamine and D-amino acid, during the forming, e.g., molding, process. Plastic-based medical devices that benefit from the present method include, but are not limited to, plastics articles used in the field of medicine, e.g. dressing materials, syringes, catheters etc., so-called “medical devices”, gloves and mattresses. Exemplary of such plastics are polypropylene, polyethylene, PVC, POM, polysulfones, polyethersulfones, polystyrenics, polyamides, polyurethanes, polyesters, polycarbonate, polyacrylics and methacrylics, polybutadienes, thermoplastic polyolefins, ionomers, unsaturated polyesters and blends of polymer resins including ABS, SAN and PC/ABS.

The polyamines, or combinations of polyamines and D-amino acids, especially in low concentrations, can be safely used even in applications where ingestion is possible, such as reusable water bottles or drinking fountains where a biofilm may develop. The surfaces of such water transport devices can be rinsed with a formulation containing one or more polyamines, or combinations of polyamines and D-amino acids, described herein, or low levels of one or more polyamines, or combinations of polyamines and D-amino acids, can be introduced into the water that passes through the containers of conduits. For example, about 0.0001% or less or up to about 1%, typically less than about 0.1% by weight of one or more polyamines, or combinations of polyamines and D-amino acids, may be introduced into such water. Given the high activity of the instant polyamines, or combinations of polyamines and D-amino acids, very small amounts are effective in many circumstances and concentrations of about 0.000001% to about 0.1%, for example, about 0.000001% to about 0.01%, or about 0.000001% to about 0.001%, or 0.000001% to 0.1%, 0.000001% to 0.01%, or 0.000001% to 0.001%, can be used in such applications.

When used in a coating or film, small amounts of one or more polyamines, or combinations of polyamines and D-amino acids, can be present for short term use, for example, one use, seasonal or disposable items, especially those applications which involve possible human contact, splints, catheters, tubing, dental equipment etc. In general, about 0.001% or less up to about 5%, for example up to about 3% or about 2%, or preferably 0.001% or less up to 5%, up to 3% or 2% by weight of one or more amino acids may be used in such coatings or films. Given the high activity of the instant polyamines, or combinations of polyamines and D-amino acids, very small amounts are effective in many circumstances and concentrations of about 0.0001% to about 1%, for example, about 0.0001% to about 0.5%, or about 0.0001% to about 0.01% can be used in coating applications, or preferably 0.0001% to 1%, 0.0001% to 0.5%, or 0.0001% to 0.01% by weight of one or more polyamines, or combinations of polyamines and D-amino acids.

For incorporation into a molded plastic article, about 0.00001% to about 10% of one or more polyamines, or combinations of polyamines and D-amino acids, can be used, for example about 0.0001% to about 3%, for example about 0.001% up to about 1% one or more polyamines, or combinations of polyamines and D-amino acids, can be used, or preferably, 0.00001% to 10%, 0.0001% to 3 0.001% up to 1% by weight one or more polyamines, or combinations of polyamines and D-amino acids, can be used. In situations in which the polyamines, or combinations of polyamines and D-amino acids, are impregnated into the surface of an already prepared molded article or fiber, the actual amount of a polyamine, or combination of a polyamine and D-amino-acid, present at the surface can depend on the substrate material, the formulation of the impregnating composition, and the time and temperature used during the impregnation step. Given the high activity of the instant polyamines, or combinations of polyamines and D-amino acids, very small amounts are effective in many circumstances, and concentrations of about 0.0001% to about 1%, for example, about 0.0001% to about 0.1%, or about 0.0001% to about 0.01% can be used in plastics, or preferably 0.0001% to 1%, 0.0001% to 0.1%, or 0.0001% to 0.01% by weight of one or more amino acids can by used.

Inhibition or reduction in a biofilm by treatment with a polyamine, or combination of a polyamine and D-amino acid, can be measured using techniques well established in the art. These techniques enable one to assess bacterial attachment by measuring the staining of the adherent biomass, to view microbes in vivo using microscopy methods; or to monitor cell death in the biofilm in response to toxic agents. Following treatment, the biofilm can be reduced with respect to the surface area covered by the biofilm, thickness, and consistency (for example, the integrity of the biofilm). Non-limiting examples of biofilm assays include microtiter plate biofilm assays, fluorescence-based biofilm assays, static biofilm assays according to Walker et al., Infect. Immun. 73:3693-3701 (2005), air-liquid interface assays, colony biofilm assays, and Kadouri Drip-Fed Biofilm assays (Merritt et al., (2005) Current Protocols in Microbiology 1.B.1.1-1.B.1.17). Such assays can be used to measure the activity of a D-amino acid on the disruption or the inhibition of formation of a biofilm (Lew et al., (2000) Curr. Med. Chem. 7(6):663-72; Werner et al., (2006) Brief Funct. Genomic Proteomic 5(1):32-6).

In other instances, treatment can be assayed by measuring the growth of bacteria and/or can be quantified by measuring the density of a biofilm-forming bacteria in a biological sample. Non-limiting examples of biological samples include blood, serum, sputum, lacrimal secretions, semen, urine, vaginal secretions, and tissue samples. The reduction in the growth of bacteria can also be measured by chest X-rays or by a pulmonary function test (PFT) (for example, spirometry or forced expiratory volume (FEV₁)).

In other situations, the presence or growth of biofilm-producing bacteria can be measured by detecting the presence of antigens of biofilm-producing bacteria in a biological sample, such as those described above. For example, an antibody to S. pneumoniae components can be used to assay colonization/infection in a subject afflicted with a biofilm-related condition or disorder, such as by assaying the presence of Streptococcus antigens in a biological sample. Such antibodies can be generated according to methods well established in the art or can be obtained commercially (for example, from Abcam, Cambridge, Mass.; Cell Sciences Canton, Mass.; Novus Biologicals, Littleton, Colo.; or GeneTex, San Antonio, Tex.).

Appropriate therapies for the treatment of biofilm-related disorders with a polyamine, or combination of a polyamine and D-amino acid, can be determined using techniques well established in the art. For example, animal models using mammals can be used to assess the efficacy of treatment with polyamines, or combinations of polyamines and D-amino acids. Non-limiting examples include implanting polymer beads, e.g., polymethylmethacrylate (PMMA) beads loaded with the polyamines, or combinations of polyamines and D-amino acids, in rats and assessing their ability to prevent biofilms.) polymethylmethacrylate (PMMA) beads in rats and catheters in rabbits have been used as animal models for biofilm formation for Staph aureus. See, e.g., Anguita-Alonzo et al., ANTIMICROBIAL AGENTS AND CHEMOTHERAPY, July 2007, p. 2594-2596, and Beenken et al. JOURNAL OF BACTERIOLOGY, July 2004, p. 4665-4684, which are hereby incorporated in its entirety by reference.

### Combination Therapy

Biofilms are understood, very generally, to be aggregations of living and dead micro-organisms, especially bacteria, that adhere to living and non-living surfaces, together with their metabolites in the form of extracellular polymeric substances (EPS matrix), e.g. polysaccharides. The activity of antibiofilm substances that normally exhibit a pronounced growth-inhibiting or lethal action with respect to planktonic cells may be greatly reduced with respect to microorganisms that are organized in biofilms, for example because of inadequate penetration of the active substance into the biological matrix.

In some instances, a polyamine, or combination of a polyamine and D-amino acid, can be administered alone or in combination with a second agent, e.g., a biocide, an antibiotic, or an antimicrobial agent, to treat a biofilm or to prevent the formation of a biofilm. An antibiotic can be co-administered with the polyamine, or combination of a polyamine and D-amino acid, either sequentially or simultaneously. For example, any of the compositions described herein can be formulated to include one or more polyamines, or combinations of polyamines and D-amino acids, and one or more second agents.

The antibiotic can be any compound known to one of ordinary skill in the art that can inhibit the growth of, or kill, bacteria. Useful, non-limiting examples of antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); glycopeptide antibiotics (such as vancomycin); polypeptide antibiotics (such as bacitracin); macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Pefloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics are commercially available, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Additional known biocides include biguanide, chlorhexidine, triclosan, chlorine dioxide, and the like.

Useful examples of antimicrobial agents include, but are not limited to, Pyrithiones, especially the zinc complex (ZPT); Octopirox®; Dimethyldimethylol Hydantoin (Glydant®); Methylchloroisothiazolinone/methylisothiazolinone (Kathon CG®); Sodium Sulfite; Sodium Bisulfite; Imidazolidinyl Urea (Germall 115®, Diazolidinyl Urea (Germain II®); Benzyl Alcohol; 2-Bromo-2-nitropropane-1,3-diol (Bronopol®); Formalin (formaldehyde); Iodopropenyl Butylcarbamate (Polyphase P100®); Chloroacetamide; Methanamine; Methyldibromonitrile Glutaronitrile (1,2-Dibromo-2,4-dicyanobutane or Tektamer®); Glutaraldehyde; 5-bromo-5-nitro-1,3-dioxane (Bronidox®); Phenethyl Alcohol; o-Phenylphenol/sodium o-phenyhphenol; Sodium Hydroxymethylglycinate (Suttocide A®); Polymethoxy Bicyclic Oxazolidine (Nuosept C®); Dimethoxane; Thimersal; Dichlorobenzyl Alcohol; Captan; Chlorphenenesin; Dichlorophene; Chlorbutanol; Glyceryl Laurate; Halogenated Diphenyl Ethers; 2,4,4′-trichloro-2′-hydroxy-diphenyl ether (Triclosan®. or TCS); 2,2′-dihydroxy-5,5′-dibromo-diphenyl ether; Phenolic Compounds; Phenol; 2-Methyl Phenol; 3-Methyl Phenol; 4-Methyl Phenol; 4-Ethyl Phenol; 2,4-Dimethyl Phenol; 2,5-Dimethyl Phenol; 3,4-Dimethyl Phenol; 2,6-Dimethyl Phenol; 4-n-Propyl Phenol; 4-n-Butyl Phenol; 4-n-Amyl Phenol; 4-tert-Amyl Phenol; 4-n-Hexyl Phenol; 4-n-Heptyl Phenol; Mono- and Poly-Alkyl and Aromatic Halophenols; p-Chlorophenol; Methyl p-Chlorophenol; Ethyl p-Chlorophenol; n-Propyl p-Chlorophenol; n-Butyl p-Chlorophenol; n-Amyl p-Chlorophenol; sec-Amyl p-Chlorophenol; Cyclohexyl p-Chlorophenol; n-Heptyl p-Chlorophenol; n-Octyl p-Chlorophenol; o-Chlorophenol; Methyl o-Chlorophenol; Ethyl o-Chlorophenol; n-Propyl o-Chlorophenol; n-Butyl o-Chlorophenol; n-Amyl o-Chlorophenol; tert-Amyl o-Chlorophenol; n-Hexyl o-Chlorophenol; n-Heptyl o-Chlorophenol; o-Benzyl p-Chlorophenol; o-Benxyl-m-methyl p-Chlorophenol; o-Benzyl-m; m-dimethyl p-Chlorophenol; o-Phenylethyl p-Chlorophenol; o-Phenylethyl-m-methyl p-Chlorophenol; 3-Methyl p-Chlorophenol; 3,5-Dimethyl p-Chlorophenol; 6-Ethyl-3-methyl p-Chlorophenol; 6-n-Propyl-3-methyl p-Chlorophenol; 6-iso-Propyl-3-methyl p-Chlorophenol; 2-Ethyl-3,5-dimethyl p-Chlorophenol; 6-sec-Butyl-3-methyl p-Chlorophenol; 2-iso-Propyl-3,5-dimethyl p-Chlorophenol; 6-Diethylmethyl-3-methyl p-Chlorophenol; 6-iso-Propyl-2-ethyl-3-methyl p-Chlorophenol; 2-sec-Amyl-3,5-dimethyl p-Chlorophenol; 2-Diethylmethyl-3,5-dimethyl p-Chlorophenol; 6-sec-Octyl-3-methyl p-Chlorophenol; p-Chloro-m-cresol: p-Bromophenol; Methyl p-Bromophenol; Ethyl p-Bromophenol; n-Propyl p-Bromophenol; n-Butyl p-Bromophenol; n-Amyl p-Bromophenol; sec-Amyl p-Bromophenol; n-Hexyl p-Bromophenol; Cyclohexyl p-Bromophenol; o-Bromophenol; tert-Amyl o-Bromophenol; n-Hexyl o-Bromophenol; n-Propyl-m,m-Dimethyl o-Bromophenol; 2-Phenyl Phenol; 4-Chloro-2-methyl phenol; 4-Chloro-3-methyl phenol; 4-Chloro-3,5-dimethyl phenol; 2,4-Dichloro-3,5-dimethylphenol; 3,4,5,6-Terabromo-2-methylphenol; 5-Methyl-2-pentylphenol; 4-Isopropyl-3-methylphenol; Para-chloro-meta-xylenol (PCMX); Chlorothymol; Phenoxyethanol; Phenoxyisopropanol; 5-Chloro-2-hydroxydiphenylmethane; Resorcinol and its Derivatives; Resorcinol; Methyl Resorcinol; Ethyl Resorcinol; n-Propyl Resorcinol; n-Butyl Resorcinol; n-Amyl Resorcinol; n-Hexyl Resorcinol; n-Heptyl Resorcinol; n-Octyl Resorcinol; n-Nonyl Resorcinol; Phenyl Resorcinol; Benzyl Resorcinol; Phenylethyl Resorcinol; Phenylpropyl Resorcinol; p-Chlorobenzyl Resorcinol; 5-Chloro 2,4-Dihydroxydiphenyl Methane; 4′-Chloro 2,4-Dihydroxydiphenyl Methane; 5-Bromo 2,4-Dihydroxydiphenyl Methane; 4′-Bromo 2,4-Dihydroxydiphenyl Methane; Bisphenolic Compounds; 2,2′-Methylene bis-(4-chlorophenol); 2,2′-Methylene bis-(3,4,6-trichlorophenol); 2,2′-Methylene bis(4-chloro-6-bromophenol); bis(2-hydroxy-3,5-dichlorophenyl)sulfide; bis(2-hydroxy-5-chlorobenzyl)sulfide; Benzoic Esters (Parabens); Methylparaben; Propylparaben; Butylparaben; Ethylparaben; Isopropylparaben; Isobutylparaben; Benzylparaben; Sodium Methylparaben; Sodium Propylparaben; Halogenated Carbanilides; 3,4,4′-Trichlorocarbanilides (Triclocarbant or TCC); 3-Trifluoromethyl-4,4′-dichlorocarbanilide; 3,3′,4-Trichlorocarbanilide; Chlorohexidine and its digluconate; diacetate and dihydrochloride; Undecenoic acid; thiabendazole, Hexetidine; poly(hexamethylenebiguanide) hydrochloride (Cosmocil®); silver compounds such as organic silver salts it anorganic silver salts, silver chloride including formulations thereof such as JM Acticare® and micronized silver particles.

### Biofilm-Related Disorders

Methods and treatments using polyamines, or combinations of polyamines and D-amino acids, include inhibiting or preventing the formation of biofilm, even or especially without inhibiting organism growth, and alos the disruption of a biofilm once formed.

A polyamine, or combination of a polyamine and D-amino acid, can be used to treat biofilm-related disorders in a subject by administering to the subject an effective amount of polyamine, or combination of a polyamine and D-amino acid, that reduces biofilm formation in the subject. A reduction in bacterial growth is indicative of the reduction in, or inhibition of, biofilm production in the subject.

In some instances, a polyamine, or combination of a polyamine and D-amino acid, can inhibit or reduce biofilm formation by diminishing adherence of biofilm-forming bacteria to a surface or by increasing bacterial death. This therapeutic approach can be useful for the treatment of biofilm-related disorders or conditions, or medical device-related infections associated with the formation of microbial biofilms.

Non-limiting examples of biofilm-related disorders include otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and cystic fibrosis. In one specific example, subjects with cystic fibrosis display an accumulation of biofilm in the lungs and digestive tract. Subjects afflicted with COPD, such as emphysema and chronic bronchitis, display a characteristic inflammation of the airways wherein airflow through such airways, and subsequently out of the lungs, is chronically obstructed.

Biofilm-related disorders can also encompass infections derived from implanted/inserted devices, medical device-related infections, such as infections from biliary stents, orthopedic implant infections, and catheter-related infections (kidney, vascular, peritoneal). An infection can also originate from sites where the integrity of the skin and/or soft tissue has been compromised. Non-limiting examples include dermatitis, ulcers from peripheral vascular disease, a burn injury, and trauma. For example, a Gram-positive bacterium, such as S. pneumoniae, can cause opportunistic infections in such tissues. The ability of S. pneumoniae to infect burn wound sites, e.g., is enhanced due to the breakdown of the skin, burn-related immune defects, and antibiotic selection.

In some instances, a subject is treated. A subject can be a mammal including, but not limited to, a primate (e.g., a monkey, such as a cynomolgous monkey, a chimpanzee, and a human). A subject can be a non-human animal such as a bird (e.g., a quail, chicken, or turkey), a farm animal (e.g., a cow, goat, horse, pig, or sheep), a pet (e.g., a cat, dog, or guinea pig, rat, or mouse), or laboratory animal (e.g., an animal model for a disorder). Non-limiting representative subjects can be a human infant, a pre-adolescent child, an adolescent, an adult, or a senior/elderly adult.

In some instances, a subject in need of treatment can be one afflicted with one or more of the infections or disorders described herein. In some instances, the subject is at risk of developing a biofilm on or in a biologically relevant surface, or already has developed such a biofilm. Such a subject at risk can be a candidate for treatment with a polyamine, or combination of a polyamine and D-amino acid, in order to inhibit the development or onset of a biofilm-production-related disorder/condition or prevent the recurrence, onset, or development of one or more symptoms of a biofilm-related disorder or condition. Such a subject can be harboring an immature biofilm that is clinically evident or detectable to the skilled artisan, but that has not yet fully formed. A subject at risk of developing a biofilm can also be one in which implantation of an indwelling device, such as a medical device, is scheduled. The risk of developing a biofilm can also be due to a propensity of developing a biofilm-related disease (such as the presence of a channel transporter mutation associated with cystic fibrosis). In such subjects, a biofilm-related disorder can be at an early stage, e.g., no bacterial infection and/or biofilm formation is yet detected.

In a specific example, the methods described herein can be used to prevent biofilm formation in the airways of a cystic fibrosis patient. Such a patient can be treated while free of bacterial infection of the airways or upon detection of a bacterial infection.

In other examples, the methods described herein can be used to prevent biofilm formation in superficial wounds of a patient. Such wounds can include burns. In some examples, the methods described herein can be used to prevent biofilm formation in patients with diabetic leg syndrome.

The invention is further described in the following example, which does not limit the scope of the invention described in the claims. Room temperature denotes a temperature from the range of 20-25° C.

### EXAMPLES

The following materials and methods were used in Examples 1-7

Strains and media.

Bacillus subtilis strains PY79, 3610 and their derivatives were grown in Luria-Bertani (LB) medium at 37° C. or MSgg medium (1) at 23° C. Solid media contained 1.5% Bacto agar. When appropriate, antibiotics were added at the following concentrations for growth of B. subtilis: 10 μg per ml of tetracycline, and 5 μg per ml of erythromycin, 500 μg per ml of spectinomycin.

Strains used:
PY79: a derivative of B. subtilis 168, was used as a host for transformation;
3610: a wild strain of B. subtilis (NCBI 3610), which is capable of forming robust biofilms (1);
Staphylococcus aureus SCOL was obtained from the Kolter lab collection (2);
E. coli strain MC4100 was obtained from the Kolter lab collection;
Pseudomonas aeruginosa PAH was obtained from the Kolter lab collection;
Strain DS76: 3610 Aeps::tet (lab stock);
Strain FC55: 3610 ΔtasA::spec (lab stock);
Strain FCS: 3610 containing P_{epsA}-lacZ at the amyE locus and a cat gene;
Strain IKG55: 3610 containing ΔracX::spec and ΔylmE::tet (3);
Strain DR30: 3610 containing tasA-mCherry at the amyE locus and a cat gene;
Strain IKG624: 3610 containing ΔyaaO::tet (this work);
Strain IKG623: 3610 containing ΔgabT::spec (this work);
Strain IKG625: 3610 containing ΔracX::mls and ΔylmE::tet, ΔgbaT::kan (this work);
Strain IKG626: 3610 containing ΔracX::spec and ΔylmE::mls, ΔyaaO::tet (this work).

Strain Construction:

Strains were constructed using standard methods (4). Long-flanking PCR mutagenesis was used to create ΔgbaT::spec and ΔyaaO (5). Primers are described in the table below. DNA was introduced into lab strains by DNA-mediated transformation of competent cells (6). SPP1 phage-mediated transduction was used to move antibiotic resistance marker-linked mutations from lab strains to the wild strain 3610 (7).

### Compounds and Reagents:

Norspermidine (1), norspermine (2), spermidine (3), spermine (4), putrescine (5), 1,3-diaminopropane (6), 1,1,5,9,9-N,N,N,N,N-pentamethylnorspermidine [N¹-(3-(dimethylamino)propyl)-N¹,N³,N³-trimethylpropane-1,3-diamine, 7], 1,1,9,9-N,N,N,N-tetramethylnorspermidine [N¹-(3-(dimethylamino)propyl)-N³, N³-dimethylpropane-1,3-diamine, (8), 5-N-methylnorspermidine [(3-aminopropyl)-N¹-methylpropane-1,3-diamine, (9), 3,3′-oxybis(propan-1-amine) (10), 3,7,11,18,22,26-Hexaazatricyclo[26.2.2.213,16]tetratriaconta-13,15,28,30,31,33-hexaene (12), N¹-dodecyl-N³-(3-(dodecylamino)propyl)propane-1,3-diamine (13), di-tert-butyl 5,5′-((3,4-dimethyl-1H-pyrrole-2,5-diyl)bis(methylene))bis(3,4-dimethyl-1H-pyrrole-2-carboxylate) (14), 2,2′-(1H-pyrrole-2,5-diyl)di(acetohydrazide) (15), 3,3′-(2,2-dimethylhydrazine-1,1-diyl)dipropanamide (16), N,N′-(azanediylbis(propane-3,1-diyl))bis(2,3,4,5,6-pentahydroxyhexanamide) (18), pentane-1,5-diamine (19), 3,3′-azanediylbis(propan-1-ol) (20), and N¹,N¹-bis(3-aminopropyl)propane-1,3-diamine (21) were obtained from Sigma-Aldrich (Atlanta, Ga.). N,N′-(azanediylbis(propane-3,1-diyl))bis(2,3,4,5,6-pentahydroxyhexanamide) (17) was purchased from Toronto research chemicals (Toronto, Canada). Diethyl 4-oxoheptanedioate (11) was synthesized according to the literature from furylacrylic acid (8). The product was purified by distillation as described previously and identified by ¹H NMR and ¹³C NMR. Texas-Red-Concanavalin A was obtained from Invitrogen-Molecular Probes (Eugene, Oreg.).

### Example 1

### Identification of Norspermidine in Conditioned Medium from B. Subtilis and the Effect of Norspermidine on Pellicle Formation

B. subtilis strain NCBI3610 was grown at 22° C. in 12-well plates in liquid biofilm-inducing medium for 3 or 8 days. Conditioned medium (500 ml) from an 8-day-old culture was concentrated on the C-18 column and eluted step-wise with methanol. Shown in FIG. 1A is the result of growing cells in fresh medium to which had been added 20 μl of the 25%, 35% or 40% methanol eluates. The 25% and 40% eluates contained compounds active in inhibiting biofilm formation whereas the 35% eluate was inert (FIG. 1A).

As reported previously, the factor in the 40% eluate was a mixture of D-amino acids (Kolodkin-Gal et al., 2010). To identify the second biofilm-inhibiting factor, high-performance liquid chromatography (HPLC) was carried out on the 25% methanol eluate using a phenyl-hexyl column. Inhibitory activity was recovered with an elution time of 40 min. Proton NMR analysis of the active fraction revealed fatty acids, morpholine and norspermidine. Cells of NCBI3610 were grown in fresh medium containing PBS buffer (control), norspermidine (100 μM), morpholine (100 μM) HPLC-purified fatty acid (−100 μM), or spermidine (100 μM). Brighter images of the norspermidine-treated cell revealed cells near the bottom of the well. Further purification using a C-18 HPLC column identified the inhibitory agent as norspermidine, a finding confirmed with authentic norspermidine, which inhibited biofilm formation at 25 uM (FIG. 1B, FIG. 2A). Pure morpholine and fatty acids detected by NMR were inactive (FIG. 1B).

Norspermidine's tendency to form strong complexes with fatty acids, which explains its elution at 25% methanol from the C-18 Sep-Pak column, complicated its quantification. To circumvent this problem we treated conditioned medium with 9-fluorenylmethyloxycarbonyl chloride (Fmoc-Cl), which protects the amino groups of norspermidine as carbamates and thereby prevents their interaction with other molecules (Molnar-Perl, 2003). Pellicles were collected from 3- and 8-day-old cultures (100 ml) of the wild type (NCBI3610) and from an 8-day-old culture (100 ml) of a gab T mutant (IKG623). After mild sonication of the pellicles, cells were separated from extracellular material. Norspermidine in the extracellular material was derivatized with Fmoc-Cl and the resulting Fmoc-norspermidine was detected using an Agilent LC/MS system. (FIG. 1C). Fmoc-norspermidine was detectable in the old pellicle from wild type cells but not in the young or mutant pellicles. See also FIG. 2B. Norspermidine was present at a concentration of 50-80 μM in 8-day-old disassembling pellicles but at a concentration of less than 1 μM in a 3-day old pellicle (FIG. 1C).

Pellicle formation of strain NCBI 3610 was tested in the presence of the indicated concentrations of norspermidine (FIG. 1D) or spermidine (FIG. 1E). FIG. 2A also shows the results of testing of pellicle formation of strain NCBI 3610 in the presence of various concentrations of norspermidine. The effect of norspermidine was specific in that a closely related polyamine, spermidine, which differs from norspermidine by the presence of an extra methylene group, was inactive in inhibiting biofilm formation at concentrations up to 2 mM (FIGS. 1D and 1E).

### Example 2

### Assessment of Biofilms when Production of D-Amino Acid and Norspermidine Production is Blocked

To evaluate the contribution of norspermidine to biofilm disassembly genetically, a mutant blocking the production of norspermidine was created. Norspermidine is synthesized from aspartate-β-semialdehyde in a pathway involving the enzyme L-diaminobutyric acid transaminase (Lee et al., 2009). A mutant lacking the B. subtilis gene (gabT) encoding this enzyme was constructed and it was found that the enzyme was blocked in norspermidine production (FIG. 1C) and was partially impaired in biofilm disassembly (FIG. 3A). The gabT mutant formed pellicles that remained relatively thick at a time (day 7) when the wild type had undergone substantial disassembly. Nonetheless, the mutant pellicle had lost the wrinkly phenotype characteristic of young biofilms by day 7.

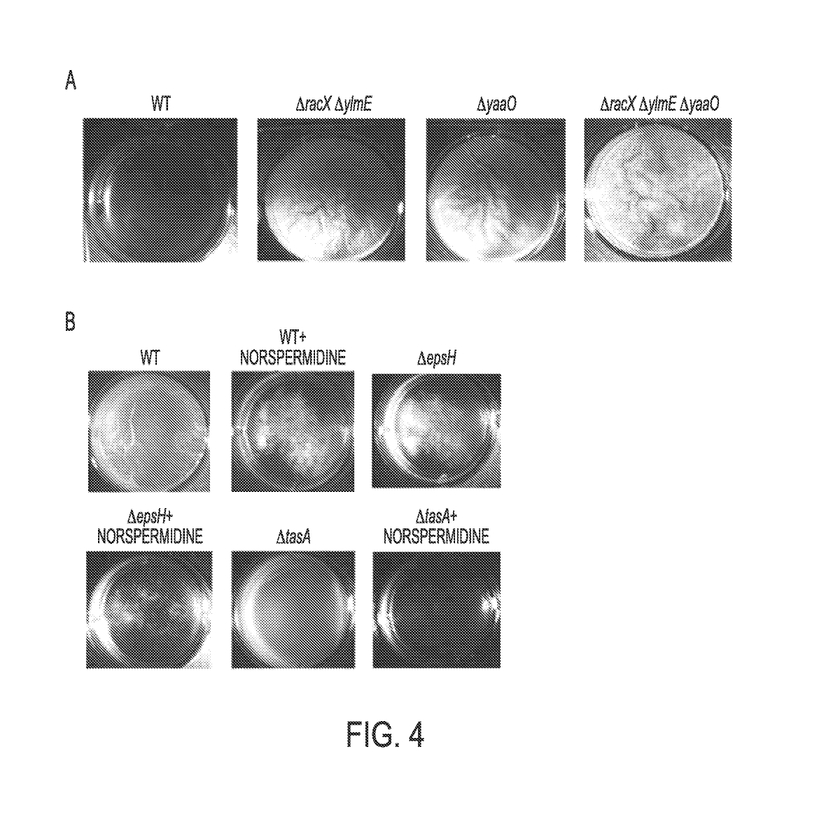

Further evaluation was conducted to assess whether the contribution of norspermidine to biofilm disassembly might be partially redundant with that of D-amino acids, which are produced by racemases encoded by racX and ylmE. Like a gabT mutant, a racX ylmE double mutant was partially impaired in biofilm disassembly. Strikingly, however, a gabT racX ylmE triple mutant formed robust pellicles that retained their wrinkly phenotype at a time (7 days) when the wild type had substantially disassembled (FIG. 3A). As a further test of the involvement of norspermidine in biofilm disassembly, a mutant lacking carboxynorspermidine decarboxylase which catalyzes the last step in the biosynthetic pathway was constructed. As in the case of the gabT mutant, a mutant lacking the B. subtilis homolog (yaaO) of the decarboxylase gene was partially impaired in biofilm disassembly and a yaaO racX ylmE triple mutant formed pellicles that remained intact at a time when the wild type had disassembled (FIG. 4A).

### Example 3

### D-Amino Acids and Norspermidine Act Through Different Mechanisms in Preventing Biofilm Formation and Triggering Biofilm Disassembly

To support the discovery that that norspermidine and D-amino acids act by different mechanisms to trigger biofilm disassembly, combinations of norspermidine with D-Tyr or with a mixture of D-Met, D-Trp, D-Leu and D-Tyr effectively prevented biofilm formation at concentrations that were ineffective in blocking biofilm formation when applied separately (FIG. 3B).

To assess whether the combination was more effective than norspermidine or D-amino acids alone, on the surface of 3 day-old pellicles were placed droplets (50 μl) containing buffer (PBS), a mixture of D-tyrosine, D-methionine, D-leucine and D-tryptophan each at final concentration of 12.5 μM, norspermidine at a final concentration of 50 μM, or, as in FIG. 3C, a combination of D-amino acids each at a concentration of only 2.5 μM and norspermidine at a concentration of 10 μM. After incubation for the indicated times, pellicle material and the medium were separated and each brought to a volume of 3 ml. After mild sonication, the OD₆₀₀ was determined for each sample. The % of disassembly represents the OD₆₀₀ of the medium as a percent of the sum of the OD₆₀₀ of the medium and the OD₆₀₀ of the pellicle. A mixture of D-amino acids and norspermidine was more effective in causing the breakdown of an existing biofilm than were either D-amino acids or norspermidine alone (FIG. 3C). Thus, D-amino acids and norspermidine act synergistically in preventing biofilm formation and triggering the disassembly of mature biofilms.

### Example 4

### Investigation of the Mechanism by which Polyamines Inhibit the Formation of and Trigger Disassembly of Biofilms

It was observed that the residual pellicle (wispy fragments of floating material with some structure) produced in the presence of norspermidine resembled pellicles seen for a mutant blocked in exopolysaccharide production but not those seen (thin, featureless pellicles) for a mutant blocked in amyloid-fiber production. More importantly, norspermidine had little effect on the residual pellicle produced by an exopolysaccharide mutant but abolished pellicle formation by the amyloid fiber mutant. In other words, the effect of norspermidine was synergistic with that of a mutation blocking fiber formation but not with a mutant blocked in exopolysaccharide production (FIG. 4B).

These observations suggested that norspermidine was interfering with the exopolysaccharide component of the matrix.

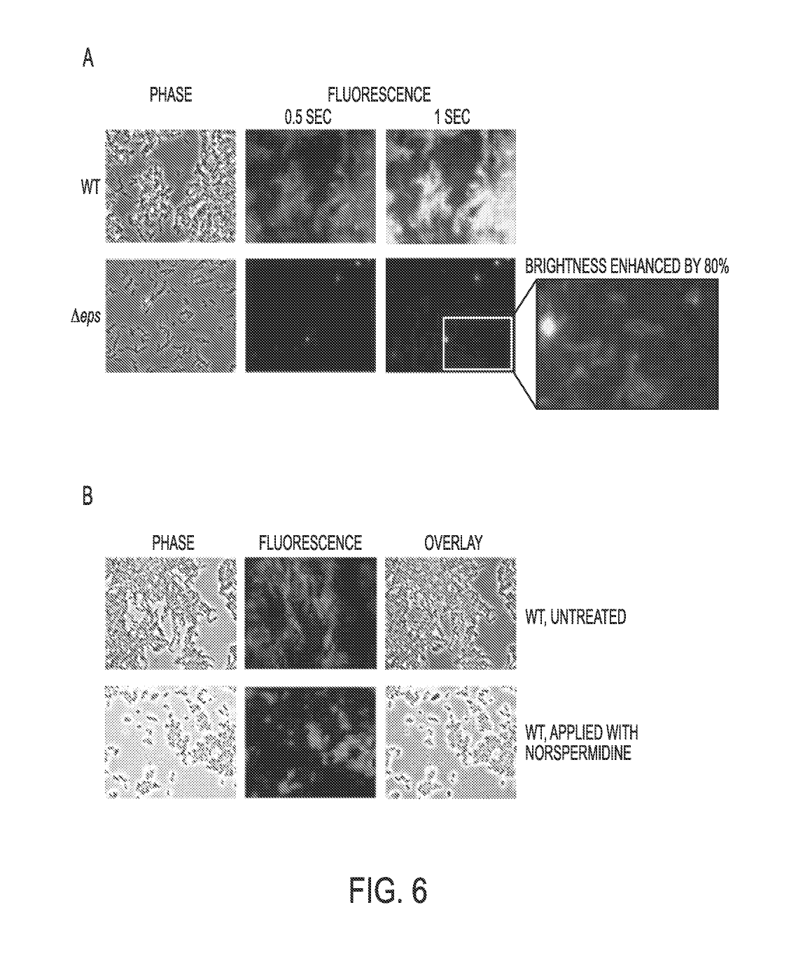

To investigate further, exopolysaccharide was visualized by fluorescence microscopy using a conjugate of the carbohydrate-binding protein concanavalin A with Texas Red (FIG. 5) (McSwain et al., 2005). As evidence of specificity, the conjugate decorated wild-type cells but not cells from a mutant (Aeps) blocked in exopolysaccharide production (FIG. 6A). Indeed, at an exposure at which concanavalin A staining with the wild type strain gave an extremely bright fluorescent signal, little or no signal could be detected for the Aeps mutant, except at long exposures and enhanced brightness (FIG. 6A). Next, we investigated the effects of norspermidine and spermidine. FIG. 5 shows that norspermidine treatment disrupted the relatively uniform, cell-associated pattern of staining seen with untreated cells, resulting in isolated patches of fluorescence. No such effect was seen with cells treated with spermidine. Simply mixing norspermidine with concanavalin A did not quench the intensity of fluorescence of the fluorophore (data not shown). Therefore, the difference in the staining was evidently due to differential levels of cell-associated exopolysaccharide. As a control, and in contrast to the results seen with concanavalin A, norspermidine had little or no effect on the protein component of the matrix as judged using a functional fusion of TasA to the fluorescent protein mCherry (FIG. 6B) (Kolodkin-Gal et al., 2010). Thus, while not being bound to any theory, it is believed that norspermidine disrupts the matrix and by targeting exopolysaccharide.

### Example 5

### Evaluation of Whether Norspermidine and Spermidine Interact with Exopolysaccharide

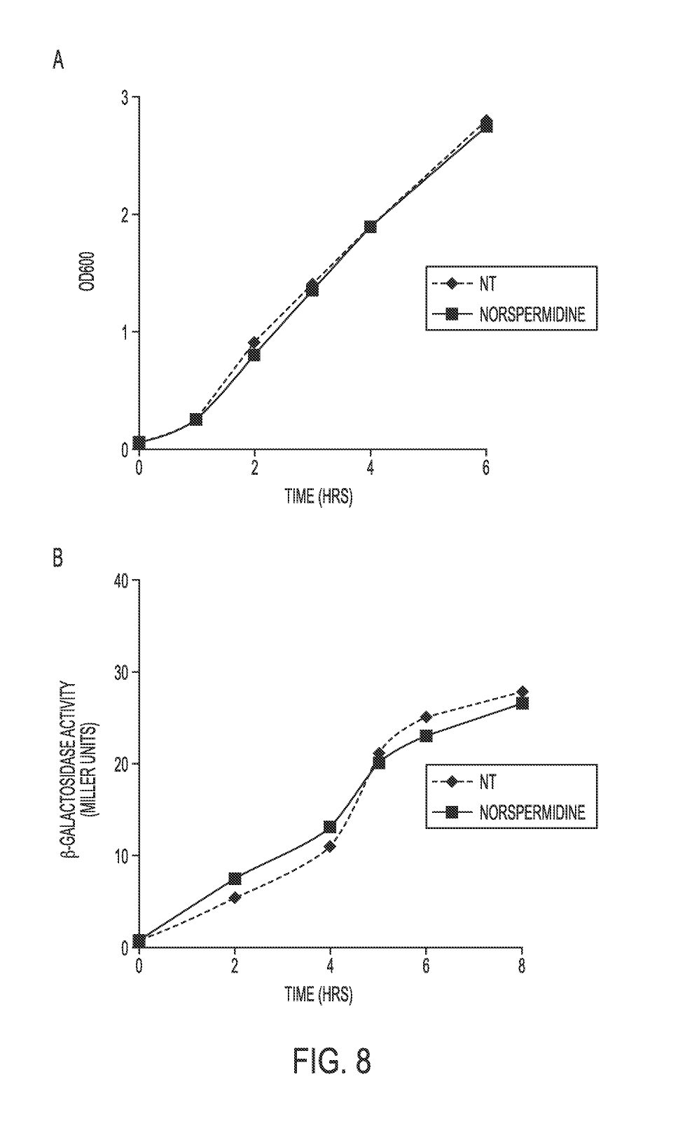

NCBI 3610 was grown in MSgg medium applied with norspermidine (100 μM) with shaking or in untreated medium served as a control (NT). The expression of the operons, epsA-O and yqxM-sipW-tasA, that specify the exopolysaccharide and protein components of the extracellular matrix, respectively, was not measurably impaired by the addition of norspermidine (FIGS. 8A & 8B). Also, cell growth was not significantly inhibited by norspermidine (FIGS. 8A & 8B). Therefore, whether norspermidine interacts directly with the exopolysaccharide was investigated. To attempt to detect such an interaction, dynamic light scattering was used. Dynamic scattering is a standard procedure for measuring the average radius of polymers in which a laser beam is transmitted through a sample containing polymers in solution (Berne, 1976; Orgad et al., 2011; Vinayahan et al., 2010).

Exopolysaccharide was purified from pellicles. Light scattering was measured for exopolysaccharide alone as well as for exopolysaccharide that had been mixed with 0.75 mM norspermidine or with 0.75 mM spermidine. Shown in FIG. 7A are the results obtained in the absence of polyamine (black), in the presence of norspermidine (white), and in the presence of spermidine (grey) with exopolysaccharide at the indicated concentrations and pH. Error bars represent the standard deviation of polymer radii among the polymers in a single sample. FIG. 7A shows that purified exopolysaccharide exhibited an average radius of 585±40 nm at pH 5.5, presumably representing an effective radius for the interacting linear polymers. Strikingly, treatment of the exopolysaccharide with norspermidine reduced the average radius substantially (175±10 nm) whereas treatment with spermidine had only a small effect on the average radius (500±20 nm). This indicates that the specificity of norspermidine resulted from a direct interaction with exopolysaccharide. The effect of norspermidine was seen over a range of exopolysaccharide concentrations (1-30 mg/ml) and also at pH 7 (FIG. 7A).

As an independent approach to detecting an interaction between norspermidine and exopolysaccharide, scanning electron microscopy was also carried out. Purified exopolysaccharide was dissolved in double distilled water at a final concentration of 10 mg/ml and mixed with either norspermidine or spermidine (0.75 mM final concentration). Samples were prepared as described. FIG. 7B shows three different magnifications of representative fields showing exopolysaccharide alone (EPS) and exopolysaccharide that had been mixed with norspermidine (EPS+norspermidine) or with spermidine (EPS+spermidine). FIGS. 8A and 8B show that controls had little effect on growth or eps transcription. Purified exopolysaccharide was seen to be in the form of aggregates, which had an average size of ˜570 nm (FIG. 7B). Strikingly, the addition of norspermidine reduced the size of the aggregates to ˜85 nm (FIG. 7B). Once again, and, as a demonstration of specificity, spermidine had little effect on the size of the aggregates.

### Example 6

### Small Molecule Screening for Biofilm-Inhibitory Activity

To identify features of norspermidine important for its biofilm disassembly activity, we tested a library of polyamines in our biofilm inhibition assay (FIGS. 9A, 9B, 10 and Table 2). In addition to norspermidine (1), we found that norspermine (2) exhibited biofilm-inhibitory activity against B. subtilis. These molecules have in common a motif consisting of three methylene groups flanked by two amino groups. The motif is present twice in 1 and three times in 2. Another polyamine, 1,3-diaminopropane (6), has only one copy of the motif and was less active (>5 mM). Also relatively inactive (inhibition was only observed at concentrations above 2 mM) were spermidine (3), spermine (4) and putrescine (5), which have a pair of amines separated by four methylenes, and cadaverine (19), which has a pair of amines separated by 5 methylenes. Replacing some or all of the amines in norspermidine with tertiary amines (7-9, 18), replacing the secondary amine with an ether linkage (10), or eliminating two or all of the amines (20, 11) resulted molecules that were relatively inactive. Whereas replacing the terminal amines with tertiary amines resulted in inactivity, in one case (21) the presence of a tertiary amine at the middle position did not block activity. The charge of each amine (at the neutral pH of the medium) was also important for biofilm inhibiting activity. Molecules that had neutral amide bonds instead of amines separated by three methylenes (15-17) were only weakly active or inactive (FIG. 10 and Table 2).

TABLE 2

| | | MBIC- |
|---|---|---|
| | | Minimal |
| | | Biofilm |
| | | Inhibitory |
| | | Concentration |
| Molecule | Formula | (μM) |
| 1 | 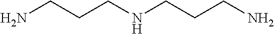 norspermidine | 25-50 |
| 2 | 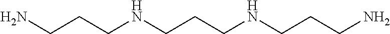 [SMILES: NCCCNCCCNCCCN] norspermine | 200 |
| 3 | 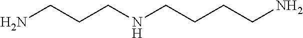 spermidine | 5000 |
| 4 | 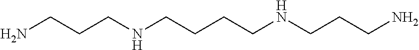 [SMILES: NCCCNCCCCNCCCN] spermine | 2500 |
| 5 | 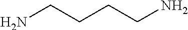 | 2000-3000 |
| 6 | 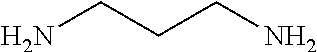 | 2500-5000 |
| 7 | 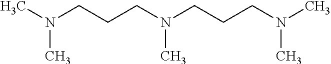 | >5000 |
| 8 | 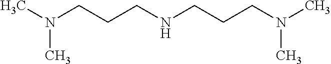 | >5000 |
| 9 | 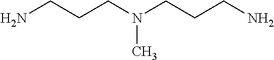 | >5000 |
| 10 | 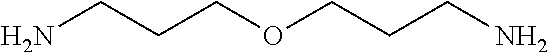 | 5000 |
| 11 | 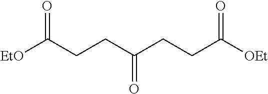 [SMILES: CCOC(=O)CCC(=O)CCC(=O)OCC] | >5000 |
| 12 | 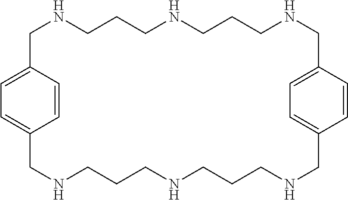 [SMILES: c1cc2ccc1CNCCCNCCCNCc1ccc(cc1)CNCCCNCCCNC2] | 500 |
| 13 | 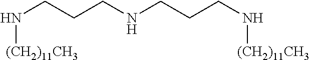 | 100 |
| 14 | 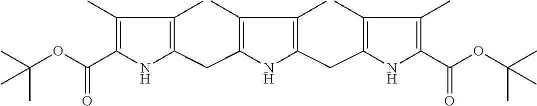 [SMILES: Cc1c(Cc2[nH]c(C(=O)OC(C)(C)C)c(C)c2C)[nH]c(Cc2[nH]c(C(=O)OC(C)(C)C)c(C)c2C)c1C] | 500-700 |
| 15 | 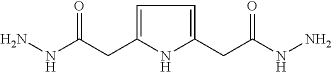 [SMILES: NNC(=O)Cc1ccc(CC(=O)NN)[nH]1] | >5000 |
| 16 | 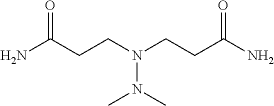 | >5000 |
| 17 | 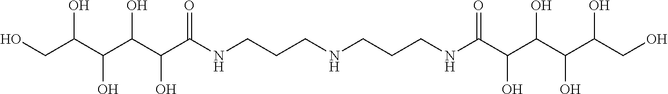 [SMILES: O=C(NCCCNCCCNC(=O)C(O)C(O)C(O)C(O)CO)C(O)C(O)C(O)C(O)CO] | >5000 |
| 18 | 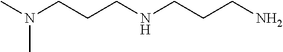 | >5000 |
| 19 | 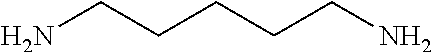 | 3000 |
| 20 | 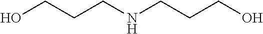 | >5000 |
| 21 | 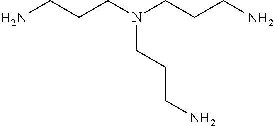 [SMILES: NCCCN(CCCN)CCCN] | 200 |

We conclude that a structure consisting of three methylene groups flanked by two positively charged amino groups contributes to biofilm-inhibiting activity. Reinforcing this hypothesis, three additional, synthetic polyamines exhibiting this motif (12, 13 and 14) were also active (FIG. 10).

FIGS. 9C, 9D, and 11 illustrate the computer modeling of the interaction of norspermidine and spermidine with an acidic exopolysaccharide. Norspermidine binds via salt bridges between amino and carboxyl groups (dotted lines) in a clamp-like mode across the exopolysaccharide secondary structure of a disaccharide repeat [α(1,6)Glc-β(1,3)GlcA]ₙ. Whereas norspermidine aligns well with the repeat, the spacing of amino groups of spermidine does not match the symmetric pattern of anionic side groups, implying weaker affinity.

### Example 7

### Evaluation of Norspermidine and Spermidine for Inhibiting Biofilm Formation by S. aureus and E. coli

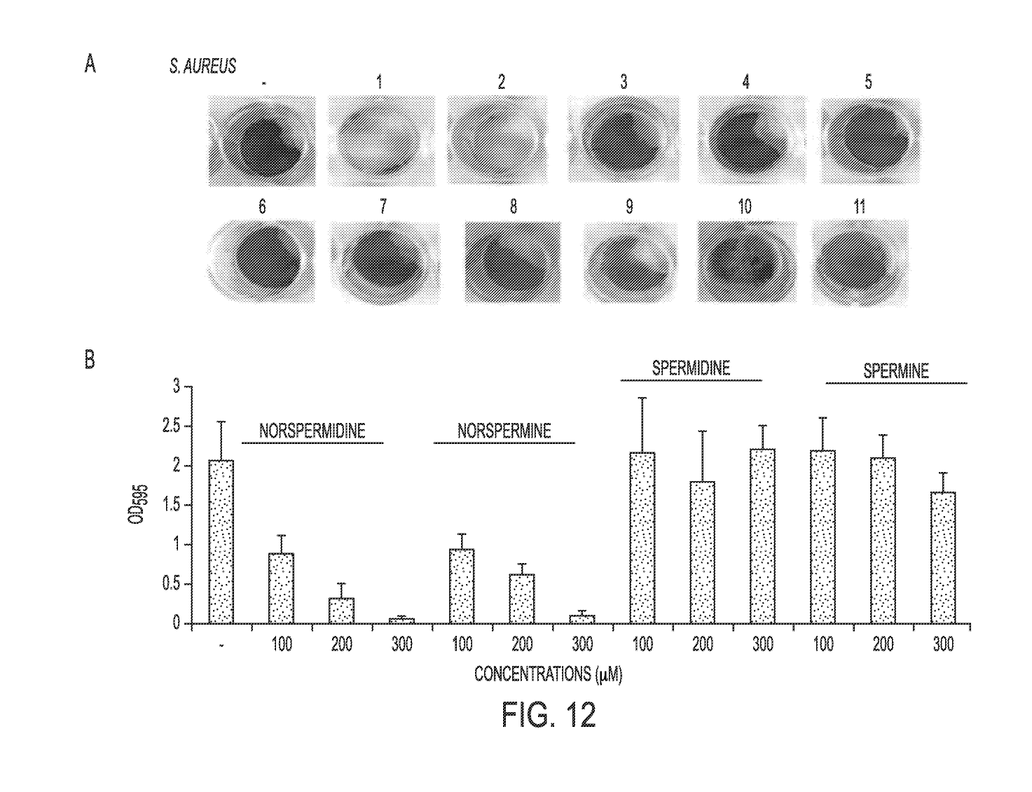

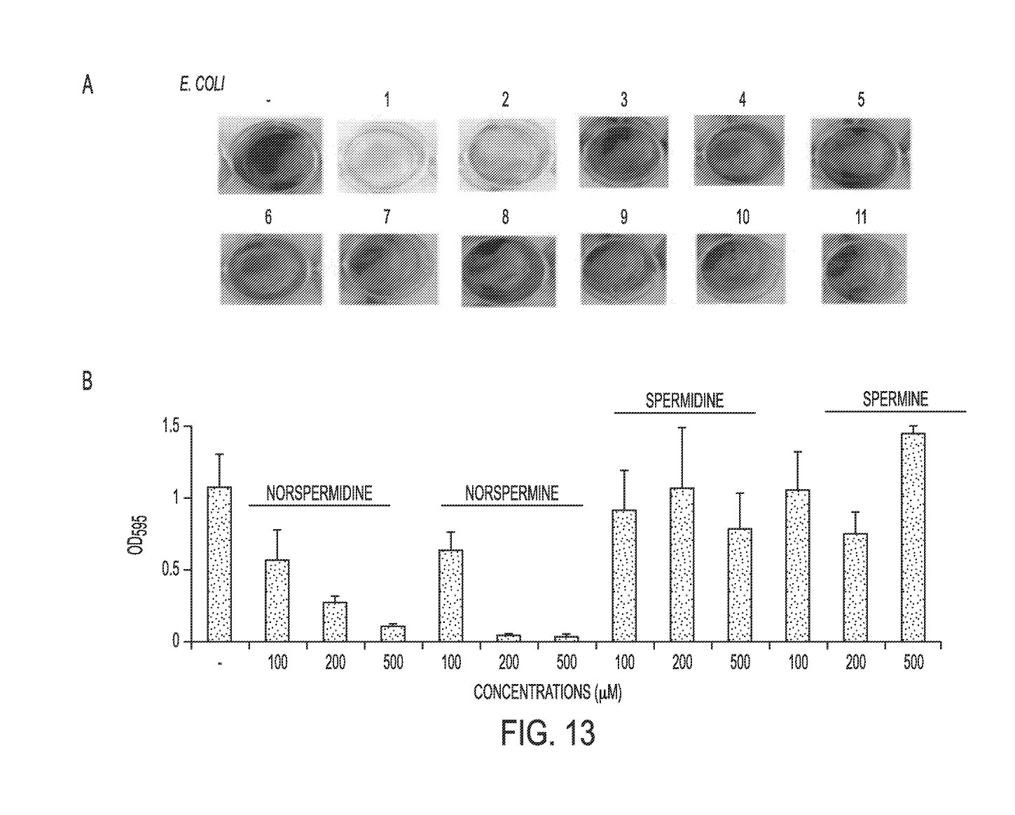

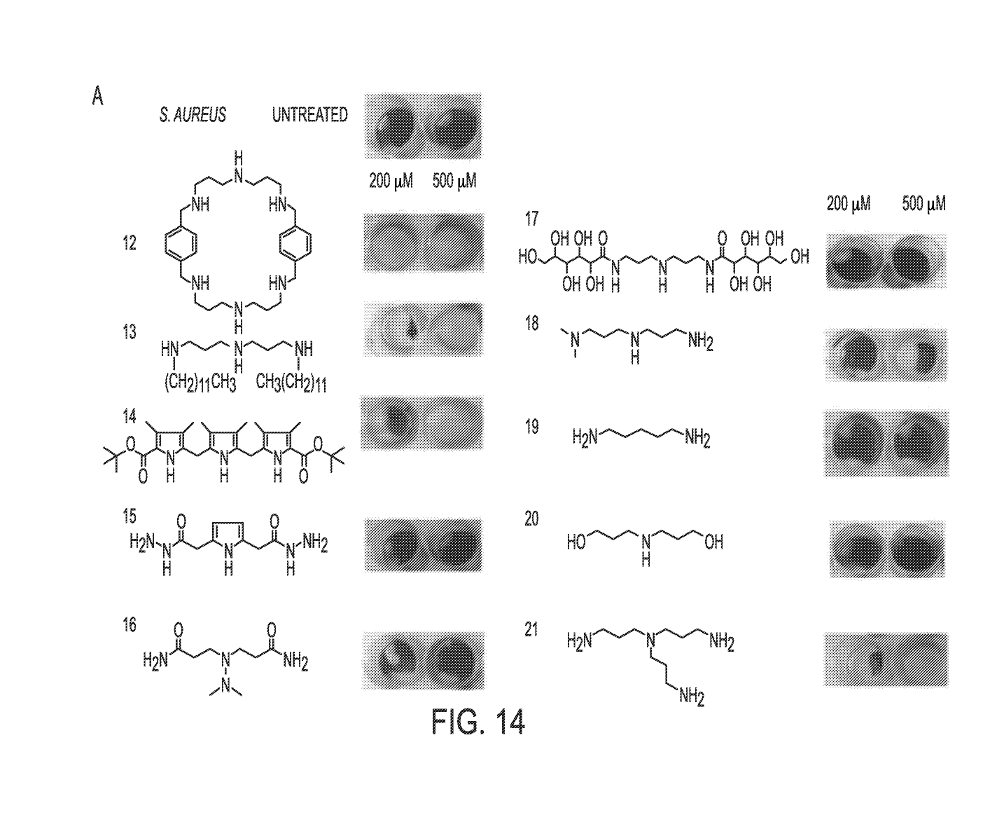

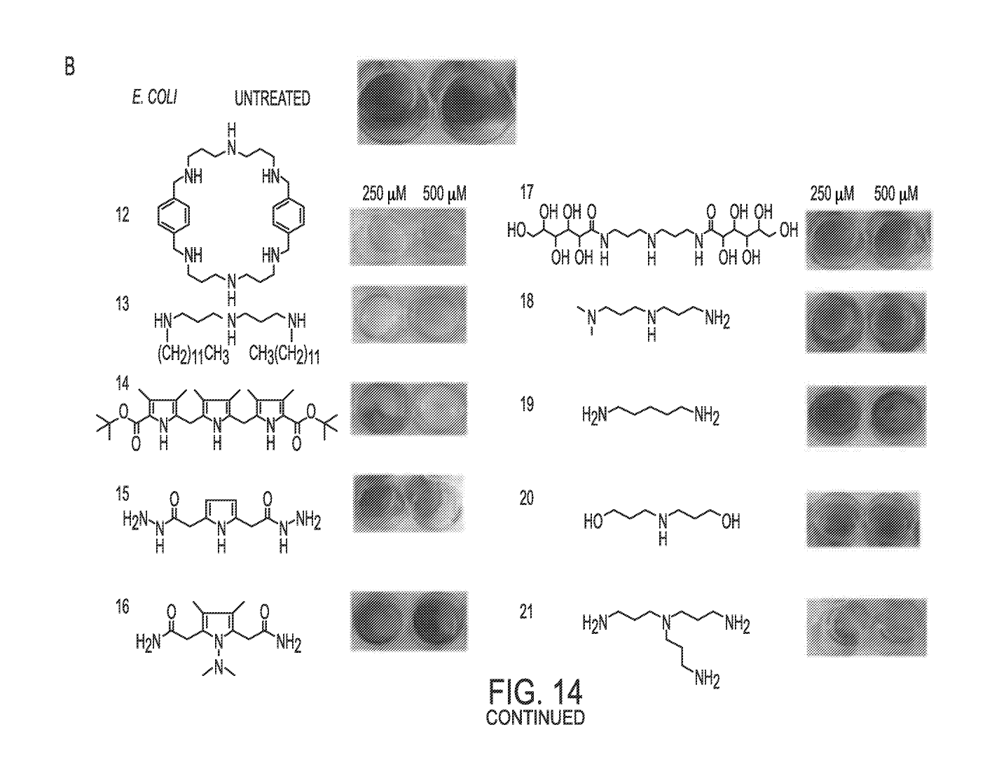

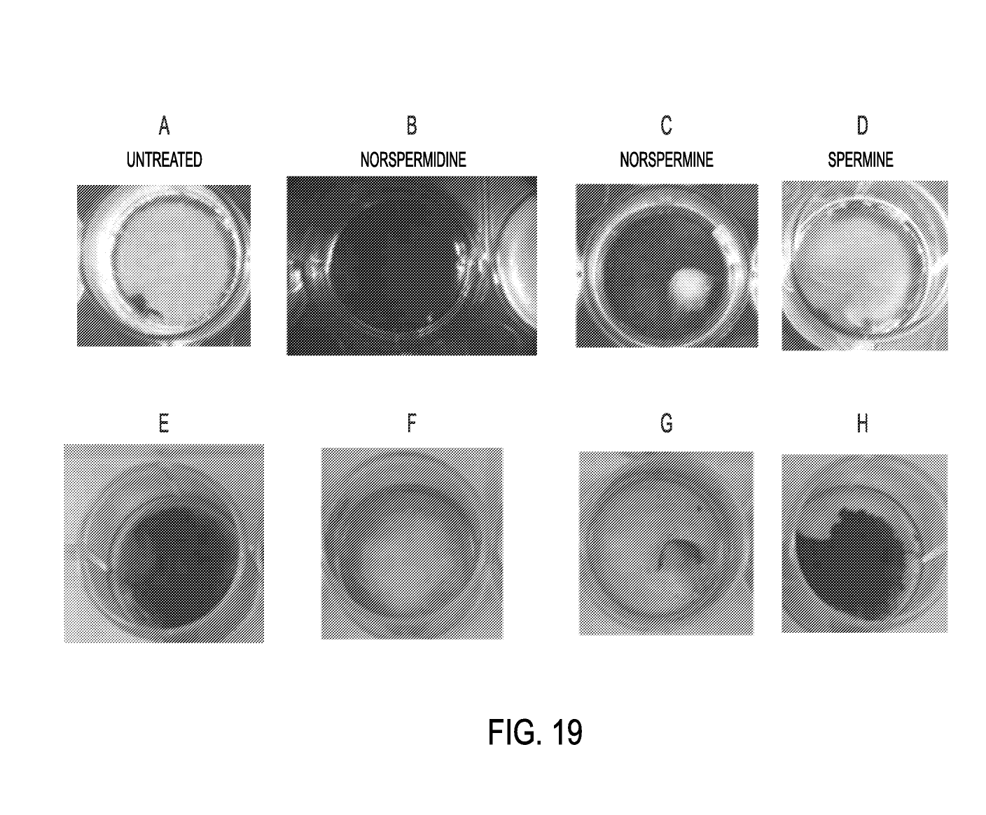

Whether polyamines might prevent biofilm formation by other bacteria that produce an exopolysaccharide matrix was examined. Indeed, the same molecules that inhibited biofilm formation by B. subtilis were effective in inhibiting biofilm formation by Staphylococcus aureus and Escherichia coli whereas those that were inactive with B. subtilis were similary not effective (FIGS. 12A & 12B, 13A & 13B, 14A & 14B, and FIGS. 19A-H). FIG. 12A shows the effect of the numbered compounds displayed in FIG. 9A on the formation of submerged biofilms by S. aureus strain SCO1. The compounds were tested at 500 μM. Biofilm formation was visualized by crystal violet staining of submerged biofilms.

Similary, FIGS. 19A-H show the results of S. aureus strain SC-01 grown to a mid-logarithmic phase and diluted 1:1000 into a 12 wells plate in a TSB medium applied with NaCl (3%) and Glucose (0.5%). The medium was applied with norspermidine (10 μM), norspermine (100 μM) or spermine (500 μM). Planktonic cells were removerd (19A-D). Wells were washed once, and incubated with Crystal Violet (0.1%) for 15 mins. Wells were washed twice in DDW (19E-H). As can be seen in FIGS. 19B, 19C, 19F, and 19G, norspermidine and norspermine inhibit biofilm formation.

In testing E. coli, the biofilm-proficient strain MC4100 was selected because a major component of exopolysacchride is colanic acid (Danese et al., 2000; Price and Raivio, 2009). Colanic acid is a negatively charged polymer, and light scattering experiments indicated a direct interaction with norspermidine. Reinforcing the idea that norspermidine was targeting the exopolysaccharide, fluorescence microscopy experiments analogous to those presented above for B. subtilis showed markedly diminished staining of exopolysaccharide when cells of S. aureus and E. coli were treated with norspermidine but not spermidine.

### Example 8

### Screening of Polyamines in Biofilm Formation in B. subtilis

B. subtilis wild-type cells were grown to a mid-logarithmic phase and 1 μl of cells was mixed with 1 μl (1 mM) of each polyamine. The mixture was plated on solid biofilm medium.

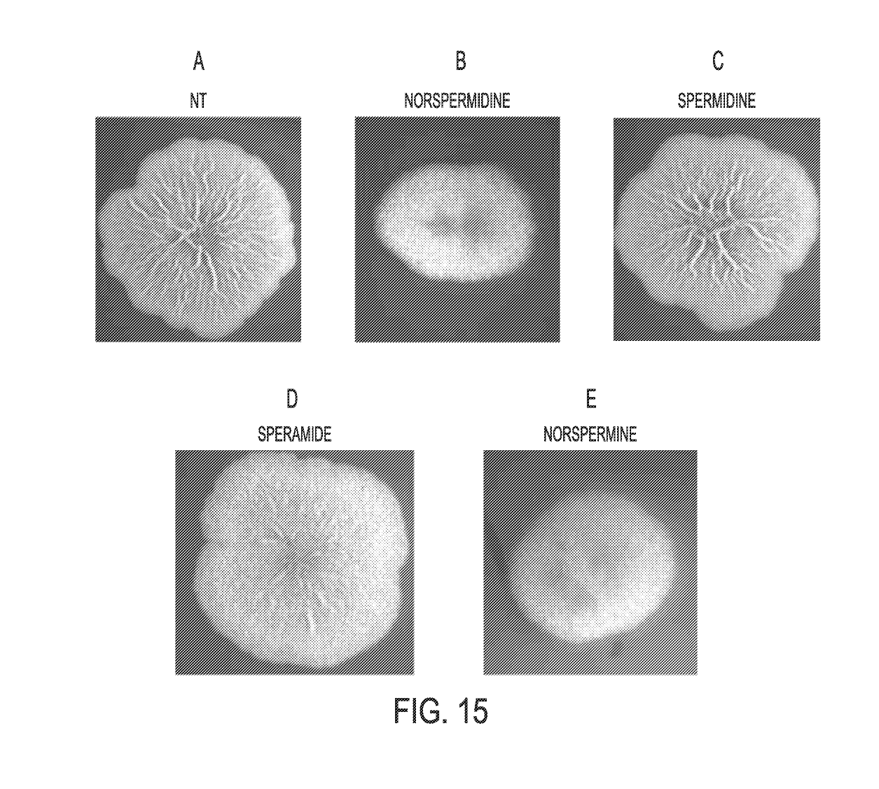

As can be seen in FIGS. 15B and 15E, norspermidine and norspermine inhibit biofilm formation. As seen in FIGS. 15C and 15D, spermidine and speramide do not.

### Example 9

### Screening of Polyamines in Pellicle Formation in B. subtilis

The effect of polyamines on pellicle formation by B. subtilis was examined. B. subtilis wild-type cells were grown to a mid-logarithmic phase and diluted 1:1000 in biofilm media applied with each polyamine to the final concentration of 50 μM.

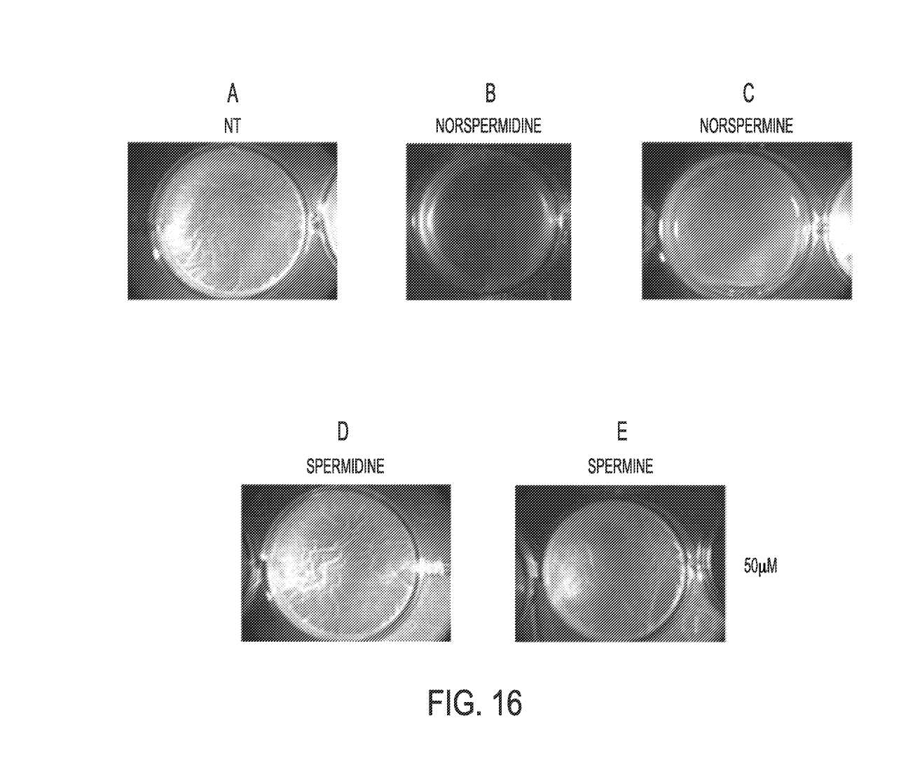

As can be seen in FIGS. 16B and 16C, norspermidine and norspermine inhibit biofilm formation. As seen in FIGS. 16D and 16E, spermidine and speramide do not.

### Example 10

### Screening of Polyamines for Disassembly of Pellicle Formation by B. subtilis

Whether polyamines could trigger disassembly of biofilms formed by B. subtilis was examined. B. subtilis wild-type cells grown in liquid biofilm medium. Cells were grown to mid-logarithmic phase and diluted 1:1000 in liquid biofilm media. At day 2, pre-formed pellicles were applied with either PBS (A) or norspermine (250 μM) (B). Pellicles were incubated for additional 24 hrs.

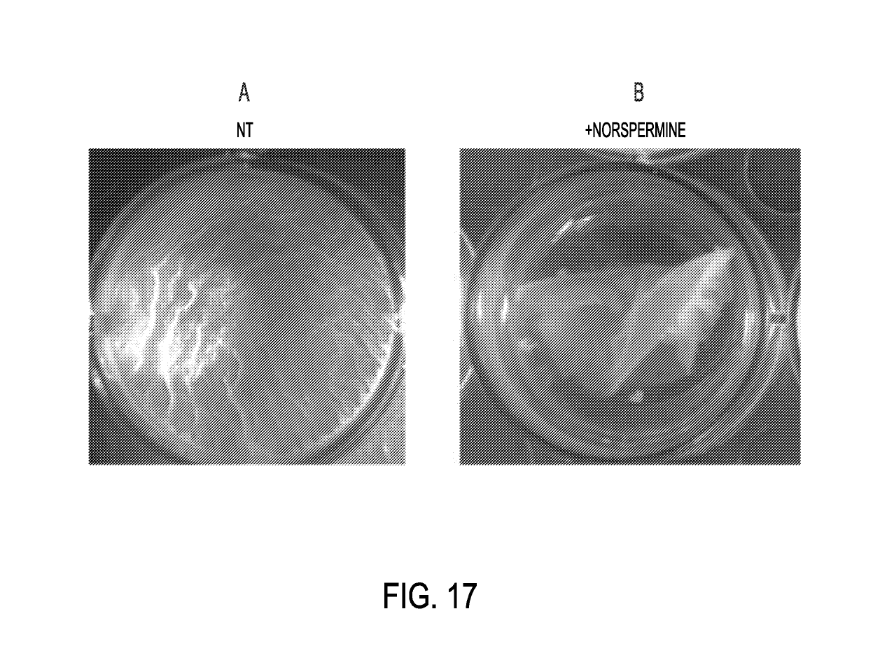

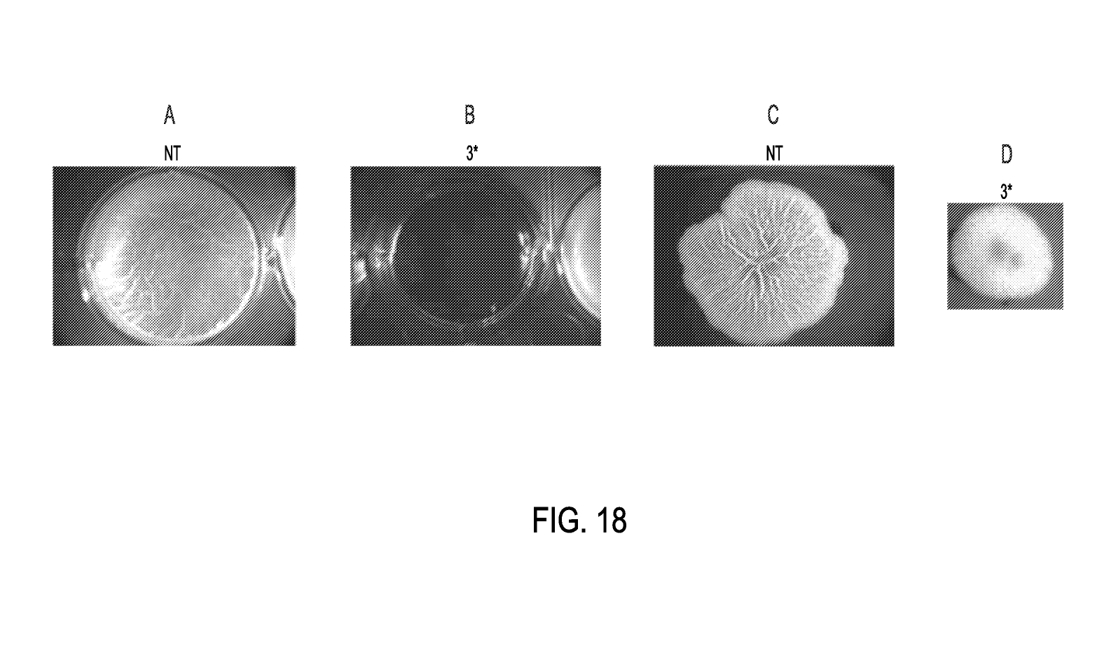

As can be seen in FIG. 17B, norspermine triggered disassembly of the pellicles.

### Example 11

### Screening of 1,5,7-Triazbicyclo[4,4,0]dec-5-ene for Biofilm Formation by B. subtilis

The effect of cyclic polyamines on biofilm formation by B. subtilis was examined. B. subtilis wild-type cells were grown to a mid-logarithmic phase and diluted 1:1000 in biofilm media (18A) or in a medium applied with cyclic compound 1,5,9-Triazacyclododecane to the final concentration of 50 μM (18B).

B. subtilis wild-type cells were grown to a mid-logarithmic phase and 1 μl of cells was either mixed with PBS (18C) or mixed with 1 μl (1 mM) of each polyamine (18D). The mixture was plated on solid biofilm medium. “3*” refers to 1,5,9-Triazacyclododecane.

As can be seen in 18B and 18D, biofilm formation was inhibited.

### Example 12

### Screening of Polyamines Combined with D-Amino Acids in Staphylococcus

The effect of certain polyamines combined with biofilm-inhibiting D-amino acids in Staphylococcus was examined. S. aureus strain SC-01 was grown to a mid-logarithmic phase and diluted 1:1000 into a 12 wells plate in a TSB medium applied with NaCl (3%) and Glucose (0.5%). The medium was applied with either norspermidine or D-tyrosine or both as indicated below each panel. Planktonic cells were removerd Wells were washed once, and incubated with Crystal Violet (0.1%) for 15 mins. Wells were washed twice in DDW.

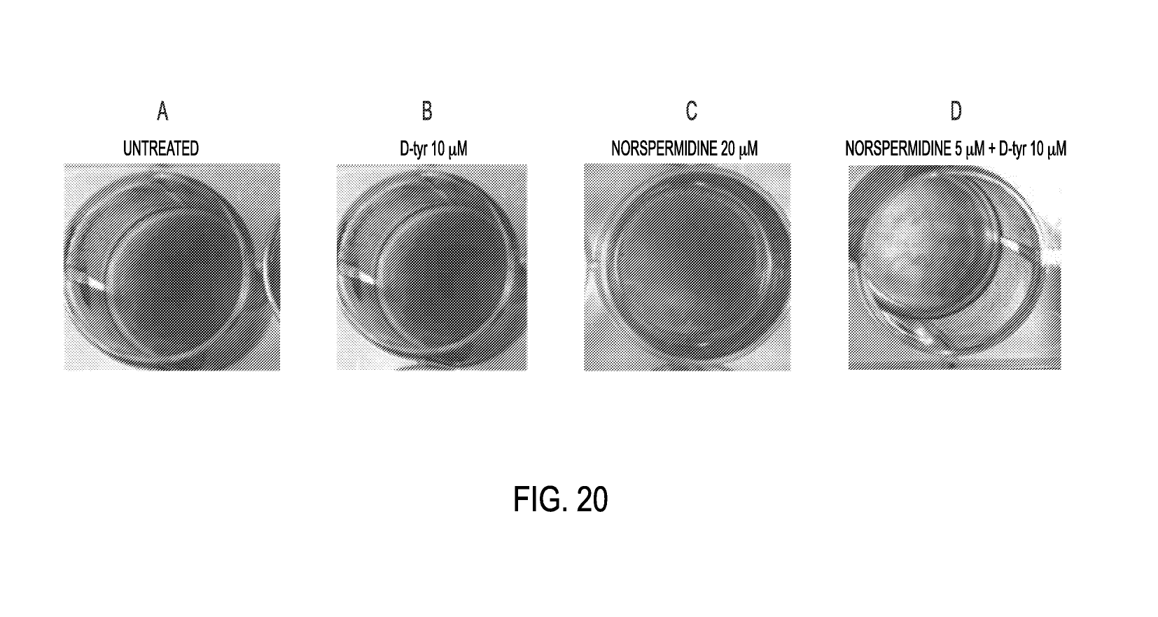

As can be seen in FIG. 20D, the combination of norspermidine and D-tyrosine acted synergistically to inhibit biofilm formation.

### Example 13

### Screening of Polyamines in Biofilm Formation in Pseudomonas

The effect of polyamines on biofilm formation by Pseudomonas was examined. P. aeriginosa strain PA14 were grown to a mid-logarithmic phase and 1 μl of cells was mixed with 1 μl (10 mM) of each polyamine. The mixture was plated on solid M63 medium.

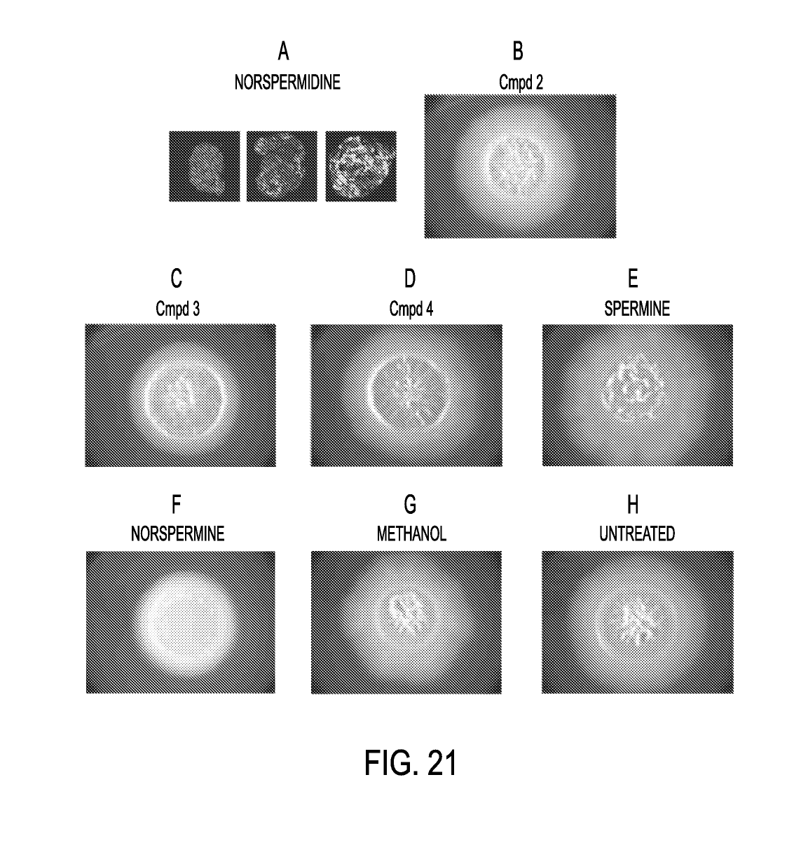

As can be seen in FIGS. 21A and 21F, norspermidine and norspermine inhibit biofilm formation.

### Example 14

### Inhibition of Proteus mirabilis Biofilm Formation by Norspermidine and Norspermine

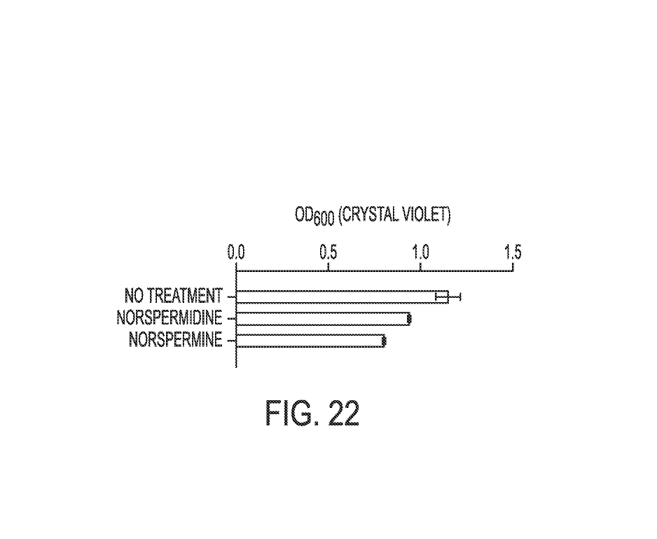

Clinically derived Proteus mirabilis strain BB2000 can form robust biofilms in multi-well polystyrene cell culture dishes and that these biofilms may be partially inhibited by 1 mM norspermidine and norspermine. To examine the effects of polyamines on biofilm formation, wells were initially treated with water (no treatment), 1 mM norspermidine, and 1 mM norspermine in 3 ml M9 + glucose before inoculation with P. mirabilis. Biofilms were permitted to grow for 48 hours at 30° C. without shaking The amount of biofilm formed was assessed with the standard assay of crystal violet staining (O'Toole G et al., Biofilm formation as microbial development, Annu Rev. Microbiol. (2000) 54:49-79); supernatant was removed from the wells and crystal violet (at 200 ml) was then incubated in each well for 15 minutes at room temperature, followed by removal of the crystal violet from the wells and a subsequent washes with water. Any crystal violet attached the wells was next solubilized with 95% ethanol for 15 minutes at room temperature, and the subsequent optical density at 595 nm in each well was measured with a multiplate reader. The addition of either 1 mM norspermidine or 1 mM norspermine was sufficient to reduce biofilm formation in comparison to the no treatment control (see figure). These results suggest that polyamines act to partially inhibit the formation of mature Proteus biofilms on polystyrene plastic surfaces. The results are shown in FIG. 22.

### Example 15

### Synthesis of Certain Amines

Certain amines of Formula (II) can be synthesized from the corresponding amides by reduction with LiAlH₄ (see e.g.: Annenkov, Synthesis of biomimetic polyamines, (2009) ARKIVOC (xiii) 116-130.). Polypropylamide, synthesized by ring opening polymerization of β-alanine N-carboxyanhydride can be reduced by BH₃SMe₂ in THF under reflux to yield the corresponding polypropylamine (Fischer, Synthesis of Linear Polyamines with Different Amine Spacings and their Ability to Form dsDNA/siRNA Complexes Suitable for Transfection, (2010) Macromol. Biosci. 10(9): 1073-1083). Another exemplary method is the hydroaminomethylation of an alkene with a primary or secondary amine with CO/H₂ and a catalyst (e.g. [Rh(cod)Cl]₂) in dioxane or toluene. Primary amines may be protected as phthalimides which are finally deprotected by hydrazinolysis in ethanol (Müller, Synthesis of polyamines via hydroaminomethylation of alkenes with urea—a new, effective and versatile route to dendrons and dendritic core molecules, (2006) Org. Biomol. Chem. 4: 826-835). Analogs of norspermine can be produced by coupling of 1,3-dibromopropanes with 1,3-diaminopropane (Kneifel, Occurrence of norspermine in Euglena gracilis, (1978) Biochem Biophys Res Comm 85(1): 42-46). Synthesis of amines of formula (II) also can be achieved from alcohols by a one-pot conversion to amines using sodium azide and triphenylphosphine in CCl₄/DMF (Reddy, A New Novel and Practical One-Pot Methodology for Conversion of Alcohols to Amines, (2000) Synth. Commun. 30(12): 2233-2237). Furthermore, norspermidine derivatives can be prepared by general synthetic methods for conversion of primary amines to secondary amines with Raney nickel using linear or branched alkyldiamines (Lee, Diamine and Triamine Analogs and Derivatives as Inhibitors of Deoxyhypusine Synthase: Synthesis and Biological Activity, (1995) J. Med. Chem. 38: 3053-3061).

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a biofilm-related disorder in a subject in need thereof, the method comprising administering to the subject a composition comprising an effective amount of a polyamine having
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—Pd]_{c} (II),
wherein
each P is R₇ or Q or L;
each L is M or —C(R₁R₂)—X—C(R₅R₆)— or 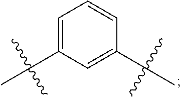 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
or a pharmaceutically acceptable salt or derivative of said polyamine,
thereby treating the biofilm-related disorder.

2. The method of claim 1, wherein the polyamine has Formula (IIa),
H₂N-(M-NH)ₓ-M-NH₂ (IIa).

3. The method of claim 1, wherein the polyamine has Formula (IIb),

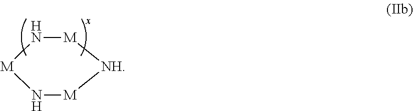

4. The method claim 1, wherein the composition is administered to a surface of the subject selected from the group of dermal and mucosal surfaces and combinations thereof.

5. The method of any of claim 1, wherein the biofilm-related disorder is selected from the group consisting of pneumonia, cystic fibrosis, otitis media, chronic obstructive pulmonary disease, and a urinary tract infection and combinations thereof.

6. The method of claim 1, wherein the polyamine is a compound or combination of compounds from the following:

| Compound | Formula |
|---|---|
| a | 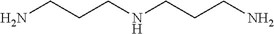 Norspermidine (also known as N-(3-aminopropyl)propane- 1,3-diamine) |
| b | 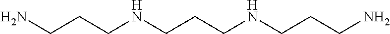 [SMILES: NCCCNCCCNCCCN] Norspermine |
| c | 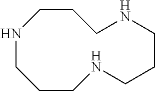 [SMILES: C1CNCCCNCCCNC1] 1,5,9-triazacyclododecane |
| d | 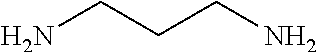 1,3-diaminopropane |
| e | 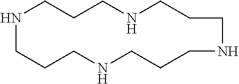 [SMILES: C1CNCCCNCCCNCCCNC1] 1,5,9,13-tetraazacyclohexadecane |
| f | 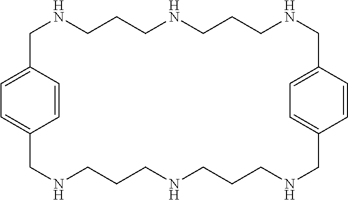 [SMILES: c1cc2ccc1CNCCCNCCCNCc1ccc(cc1)CNCCCNCCCNC2] 3,7,11,18,22,26-Hexaazatricyclo[26.2.2.213,16] tetratriaconta-13,15,28,30,31,33-hexaene |
| g | 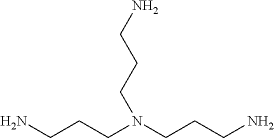 [SMILES: NCCCN(CCCN)CCCN] N¹,N¹-bis(3-aminopropyl)propane-1,3-diamine |
| h | 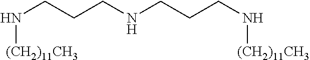 N1-dodecyl-N3-(3-(dodecylamino)propyl)propane- 1,3-diamine |
| i | 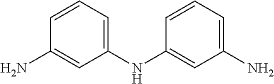 [SMILES: Nc1cccc(Nc2cccc(N)c2)c1] N,N-Di(3-aminophenyl)amine |

7. A method of treating, reducing, or inhibiting biofilm formation by bacteria on a biologically-related surface, the method comprising:
contacting a biological surface with a composition comprising an effective amount of a polyamine having:
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—Pd]_{c} (II),
wherein
each P is R₇ or Q or L;
each L is M or —C(R₁R₂)—X—C(R₅R₆)— or 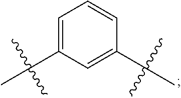 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
thereby treating, reducing, or inhibiting formation of the biofilm.

8. The method of claim 7, wherein the polyamine has Formula (IIa),
H₂N-(M-NH)ₓ-M-NH₂ (IIa).

9. The method of claim 7, wherein the polyamine has Formula (IIb),

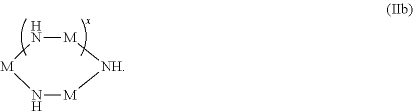

10. The method claim 7, wherein the surface comprises a medical device, a wound dressing, a contact lens, or an oral device.

11. The method of claim 7, wherein the polyamine is a compound or combination of compounds from the following:

| Compound | Formula |
|---|---|
| a | 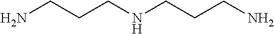 Norspermidine (also known as N-(3-aminopropyl)propane- 1,3-diamine) |
| b | 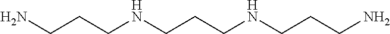 [SMILES: NCCCNCCCNCCCN] Norspermine |
| c | 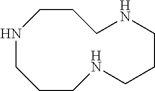 [SMILES: C1CNCCCNCCCNC1] 1,5,9-triazacyclododecane |
| d | 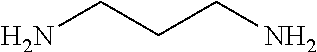 1,3-diaminopropane |
| e | 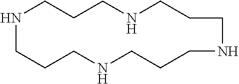 [SMILES: C1CNCCCNCCCNCCCNC1] 1,5,9,13-tetraazacyclohexadecane |
| f | 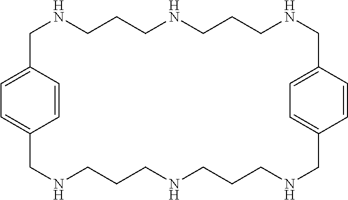 [SMILES: c1cc2ccc1CNCCCNCCCNCc1ccc(cc1)CNCCCNCCCNC2] 3,7,11,18,22,26-Hexaazatricyclo[26.2.2.213,16] tetratriaconta-13,15,28,30,31,33-hexaene |
| g | 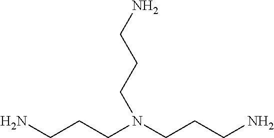 [SMILES: NCCCN(CCCN)CCCN] N¹,N¹-bis(3-aminopropyl)propane-1,3-diamine |
| h | 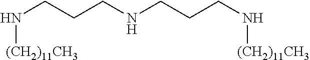 N1-dodecyl-N3-(3-(dodecylamino)propyl)propane- 1,3-diamine |
| i | 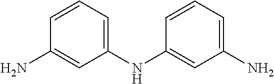 [SMILES: Nc1cccc(Nc2cccc(N)c2)c1] N,N-Di(3-aminophenyl)amine |

12. The method of claim 7, wherein the composition further comprises a D-amino acid, or pharmaceutically acceptable salts, esters, or derivatives thereof.

13. The method of claim 12,
wherein the composition comprises a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

14. A composition comprising:
a pharmaceutically acceptable carrier, a cosmetically suitable base, or an orally acceptable carrier; and
a polyamine having
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—Pd]_{c} (II),
wherein
each P is R₇ or Q or L;
each L is M or —C(R₁R₂)—X—C(R₅R₆)— or 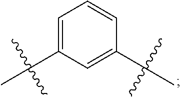 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
in an amount effective to treat, reduce, or inhibit biofilm formation.

15. The composition of claim 14, wherein the polyamine has Formula (IIa),
H₂N-(M-NH)ₓ-M-NH₂ (IIa).

16. The composition of claim 14, wherein the polyamine has Formula (IIb),

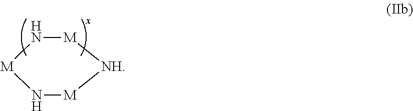

17. The composition of claim 14, wherein the polyamine is a compound or a combination of compounds from the following

| Compound | Formula |
|---|---|
| a | 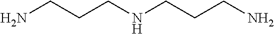 Norspermidine (also known as N-(3-aminopropyl)propane- 1,3-diamine) |
| b | 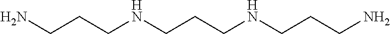 [SMILES: NCCCNCCCNCCCN] Norspermine |
| c | 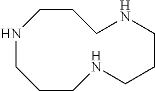 [SMILES: C1CNCCCNCCCNC1] 1,5,9-triazacyclododecane |
| d | 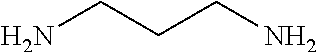 1,3-diaminopropane |
| e | 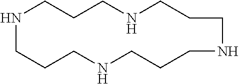 [SMILES: C1CNCCCNCCCNCCCNC1] 1,5,9,13-tetraazacyclohexadecane |
| f | 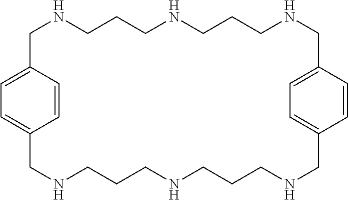 [SMILES: c1cc2ccc1CNCCCNCCCNCc1ccc(cc1)CNCCCNCCCNC2] 3,7,11,18,22,26-Hexaazatricyclo[26.2.2.213,16] tetratriaconta-13,15,28,30,31,33-hexaene |
| g | 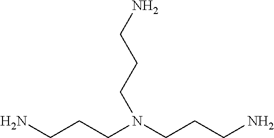 [SMILES: NCCCN(CCCN)CCCN] N¹,N¹-bis(3-aminopropyl)propane-1,3-diamine |
| h | 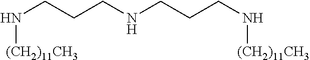 N1-dodecyl-N3-(3-(dodecylamino)propyl)propane- 1,3-diamine |
| i | 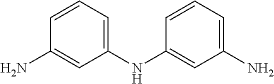 [SMILES: Nc1cccc(Nc2cccc(N)c2)c1] N,N-Di(3-aminophenyl)amine |

18. A biofilm resistant medical device, comprising:
a surface likely to contact a biological fluid, said surface comprising a composition of claim 14 coated on or impregnated into said surface, wherein the polyamine is present in an amount effective to treat, reduce, or inhibit biofilm formation.

19. The composition of claim 14, wherein the composition is selected from the group consisting of shampoos, bath additives, hair care preparations, soaps, lotions, creams, deodorants, skin-care preparations, cosmetic personal care preparations, intimate hygiene preparations, foot care preparations, light protective preparations, skin tanning preparations, insect repellants, antiperspirants, sharing preparations, hair removal preparations, fragrance preparations, dental care, denture care and mouth care preparations and combinations thereof.

20. The composition of claim 14, wherein the composition is an oral composition in the form of a toothpaste, tooth gel, tooth powder, mouthwash, mouth rinse, mouth spray, a dental solution, or an irrigation fluid.
